SEQUENCE LISTING (7 sequences)

SEQ ID NO: 1 - DNA, 26 bp, Artificial
  Primer P1
  primer_bind (1)..(26)
tgcatctacagggttaccatggagaa

SEQ ID NO: 2 - DNA, 29 bp, Artificial
  Primer P2
  primer_bind (1)..(29)
tatccctgcaggccttcagcagaggctct

SEQ ID NO: 3 - DNA, 28 bp, Artificial
  Primer P3
  primer_bind (1)..(28)
ttcacatggatatgcgacggtaccttct

SEQ ID NO: 4 - DNA, 31 bp, Artificial
  Primer P4
  primer_bind (1)..(31)
ctgtgaagaagaactatcggcgggttcctta

SEQ ID NO: 5 - DNA, 967 bp, pig
  misc_feature (1)..(967): pig priming with P1 and P2
tgcatctacagggttaccatggagaagctgtcctaccacagcatttgtaccgcggaagagtggcaaggcctcatgcgcttcaaccttcccgtccgtctttgcaaggagattgaattgttccacttcgacattggtccttttgaaaacatgtggcctggaatctttgtctatatggttcatcgcttctgtgggacggcctgctttgagcttgaaaagctgtgtcgttttatcatgtctgtgaagaagaactatcgtcgggttccttaccacaactggaagcacgcggtcacggtggcacactgcatgtacgccatcctccagaacagccacgggctcttcaccgacctcgagcgcaaaggactgctaatcgcgtgtctgtgccacgacctggaccacaggggcttcagcaacagctacctgcagaaattcgaccaccccctggccgctctctactccacgcccaccatggagcagcaccacttctcccagaccgtgtccatcctccagttggaagggcacaacatcttctccaccctgagctccagtgagtacgagcaggtgcttgagatcatccgcaaagccatcattgccacagacctcgctttgtactttggaaacaggaaacagttggaggagatgtaccagaccggatcgctaaaccttaataaccagtcacatagagaccgcgtcattggtttgatgatgactgcctgtgatctctgttccgtgacaaaactgtggccagtaacaaaactgacggcaaatgatatatatgcggaattctgggccgagggcgatgaggtgaagaagctgggaatacagcctattcccatgatggacagagacaagaaggacgaagtcccacaaggccagctcggattctacaacgcggtagctatcccctgctacaccaccctcacccagatcttcccgcccacagagcctcttctgaaggcctgcagggata

SEQ ID NO: 6 - DNA, 732 bp, guinea pig
  misc_feature (1)..(732): guinea pig priming with P4 and P2
ctgtgaagaagaactatcggcgggttccttaccacaactggaagcatgcagtcacggtggcgcactgcatgtacgccatacttcaaaacaacaatggcctcttcacagaccttgagcgcaaaggcctgctaattgcctgtctgtgccatgacctggaccacaggggcttcagtaacagctacctgcagaaattcgaccaccccctggctgcgttgtactccacctccaccatggagcaacaccacttctcccagacggtgttcatcctccagctggaaggacacaacatcttctccaccctgagctccagcgagtacgagcaggtgctggagatcatccgcaaagccatcatcgccactgacctcgcactgtactttgggaacaggaagcagttggaggagatgtaccagacagggtcgctgaacctcaataaccagtcccatcgagaccgcgtcatcggcttgatgatgactgcctgcgatctttgctctgtgacgaaactatggccagttacaaaattgacagcaaatgatatatatgcagagttctgggctgagggggatgagatgaagaagttggggatacagcccatccctatgatggacagagacaagaaggatgaagtccctcaaggacagcttggattctacaatgctgtggccatcccctgctataccaccctgacgcagatcctcccacccacagagcctctgctgaaggcctgcagggata

SEQ ID NO: 7 - DNA, 266 bp, guinea pig
  misc_feature (1)..(266): guinea pig priming with P2 and P3
tagagcctctgctgaaggcctgcagggataacctcaatcagtgggagaaggtaattcgaggggaagagacagcaatgtggatttcaggcccagcaactagcaaaagcacatcagggaagccgaccaggaaggtcgatgactgatcctgaggtgatgtctgcctagcaactgactcaacctgcttctgtgacttcgttctttttatttttatttttttaacggggtgaaaacctctctcagaaggtaccgtcgcatatccatgtgaa

(19) United States
(12) Patent Application Publication
(10) Pub. No.: US 2007/0299079 A1
(43) Pub. Date: Dec. 27, 2007

(54) 4-AMINO-PYRIDO[3,2-e]PYRAZINES, THEIR USE AS INHIBITORS OF PHOSPHODIESTERASE 10, AND PROCESSES FOR PREPARING THEM

(76) Inventors: Hofgen Norbert, Ottendorf-Okrilla (DE); Stange Hans, Riesa (DE); Langen Barbara, Radebeul (DE); Egerland Ute, Radebeul (DE); Schindler Rudolf, Dresden (DE); Gasparic Antje, Coswig (DE); Rundfeldt Chris, Coswig (DE)

Correspondence Address: FULBRIGHT & JAWORSKI, LLP, 666 FIFTH AVE, NEW YORK, NY, 10103-3198, US

(21) Appl. No.: 11/753,260

(22) Filed: May 24, 2007

Related U.S. Application Data
(60) Provisional application No. 60/809,251, filed on May 30, 2006.

(51) Int. Cl. A61K 31/4985; A61P 25/00; C07D 471/14

(52) U.S. Cl. 514/250; 544/346

(57) ABSTRACT

The invention relates to 4-amino-pyrido[3,2-e]pyrazines, to processes for preparing them, to pharmaceutical preparations which comprise these compounds and to the pharmaceutical use of these compounds, which are inhibitors of phosphodiesterase 10, as active compounds for treating diseases of mammals including a human which can be influenced by using the compounds according to the invention to inhibit phosphodiesterase 10 activity in the central nervous system. More particularly, the invention relates to the treatment of neurologic and psychiatric disorders, for example psychosis and disorders comprising cognitive deficits as symptoms.

The invention relates to 4-amino-pyrido[3,2-e]pyrazines, to processes for preparing them, to pharmaceutical preparations which comprise these compounds and to the pharmaceutical use of these compounds, which are inhibitors of phosphodiesterase 10, as active compounds for treating diseases of mammals including a human which can be influenced by using the compounds according to the invention to inhibit phosphodiesterase 10 activity in the central nervous system. More particularly, the invention relates to the treatment of neurologic and psychiatric disorders, for example psychosis and disorders comprising cognitive deficits as symptoms.

### BACKGROUND

Psychotic disorders, especially schizophrenia, are severe mental disorders which extremely impair daily life. The symptoms of psychosis may be divided into two fractions. In the acute phase, it is predominated by hallucinations and delusions being called the positive symptoms. When the agitated phase abates the so called negative symptoms become obvious. They include cognitive deficits, social phobia, reduced vigilance, indifference and deficits in verbal learning and memory, verbal fluency and motor function.

Although several antipsychotics are available since, the present therapy of psychosis is not satisfactory. The classic antipsychotics, such as haloperidol, with a high affinity to dopamine D2 receptor show extreme side effects, such extrapyramidal symptoms (=EPS) and do not improve the negative symptoms of schizophrenia so that they do not enable the patient to return to everyday life.

Clozapine which has emerged as a benchmark therapeutic ameliorating positive, negative and cognitive symptoms of schizophrenia and devoid of EPS shows agranulocytosis as a major, potential lethal side-effect (Capuano et al., 2002). Besides, there is still a high amount of therapy resistant cases (Lindenmayer et al., 2002).

In conclusion, there is still a need for developing new antipsychotics which ameliorate positive, negative and cognitive symptoms of psychosis and have a better side effect profile.

The exact pathomechanism of psychosis is not yet known. A dysfunction of several neurotransmitter systems has been shown. The two major neurotransmitter systems that are involved are the dopaminergic and the glutamatergic system:

Thus, acute psychotic symptoms may be stimulated by dopaminergic drugs (Capuano et al., 2002) and classical antipsychotics, like haloperidol, have a high affinity to the dopamine D2 receptor (Nyberg et al., 2002). Animal models based on a hyperactivity of the dopaminergic neurotransmitter system (amphetamine hyperactivity, apomorphine climbing) are used to mimic the positive symptoms of schizophrenia.

Additional there is growing evidence that the glutamatergic neurotransmitter system plays an important role in the development of schizophrenia (Millan, 2005). Thus, NMDA antagonists like phencyclidine and ketamine are able to stimulate schizophrenic symptoms in humans and rodents (Abi-Saab et al., 1998; Lahti et al., 2001). Acute administration of phencyclidine and MK-801 induce hyperactivity, stereotypies and ataxia in rats mimicking psychotic symptoms. Moreover, in contrast to the dopaminergic models the animal models of psychosis based on NMDA antagonists do not only mimic the positive symptoms but also the negative and cognitive symptoms of psychosis (Abi-Saab et al., 1998; Jentsch and Roth, 1999). Thus, NMDA antagonists, additionally induce cognitive deficits and social interaction deficits.

Eleven families of phosphodiesterases have been identified in mammals so far (Essayan, 2001). The role of PDEs in the cell signal cascade is to inactivate the cyclic nucleotides cAMP and/or cGMP (Soderling and Beavo, 2000). Since cAMP and cGMP are important second messenger in the signal cascade of G-protein-coupled receptors PDEs are involved in a broad range of physiological mechanisms playing a role in the homeostasis of the organism.

The PDE families differ in their substrate specificity for the cyclic nucleotides, their mechanism of regulation and their sensitivity to inhibitors. Moreover, they are differentially localized in the organism, among the cells of an organ and even within the cells. These differences lead to a differentiated involvement of the PDE families in the various physiological functions.

PDE10A is primarily expressed in the brain and here in the nucleus accumbens and the caudate putamen. Areas with moderate expression are the thalamus, hippocampus, frontal cortex and olfactory tubercle (Menniti et al., 2001). All these brain areas are described to participate in the pathomechanism of schizophrenia (Lapiz et al. 2003) so that the location of the enzyme indicates a predominate role in the pathomechanism of psychosis.

In the striatum PDE10A is predominately found in the medium spiny neurons and there are primarily associated to the postsynaptic membranes of these neurons (Xie et al., 2006). By this location PDE10A may have an important influence on the signal cascade induced by dopaminergic and glutamatergic input on the medium spiny neurons two neurotransmitter systems playing a predominate role in the pathomechanism of psychosis.

Phosphodiesterase (PDE) 10A, in particular, hydrolyses both cAMP and cGMP having a higher affinity for cAMP (Kₘ=0.05 μM) than for cGMP (K_{M}=3 μM) (Soderling et al., 1999).

Psychotic patients have been shown to have a dysfunction of cGMP and cAMP levels and its downstream substrates (Kaiya, 1992; Muly, 2002; Garver et al., 1982). Additionally, haloperidol treatment has been associated with increased cAMP and cGMP levels in rats and patients, respectively (Leveque et al., 2000; Gattaz et al., 1984). As PDE10 hydrolyses both cAMP and cGMP (Kotera et al., 1999) an inhibition of PDE10A would also induce an increase of cAMP and cGMP and thereby having a similar effect on cyclic nucleotide levels as haloperidol.

The antipsychotic potential of PDE10A inhibitors is further supported by studies of Kostowski et al. (1976) who showed that papaverine, a moderate selective PDE10A inhibitor, reduces apomorphine-induced stereotypies in rats, an animal model of psychosis, and increases haloperidol-induced catalepsy in rats while concurrently reducing dopamine concentration in rat brain. Activities that are also seen with classical antipsychotics. This is further supported by a patent application establishing papaverine as a PDE10A inhibitor for the treatment of psychosis (US Patent Application No. 2003/0032579).

In addition to classical antipsychotics which mainly ameliorate the positive symptoms of psychosis PDE10A also bears the potential to improve the negative and cognitive symptoms of psychosis.

Focusing on the dopaminergic input on the medium spiny neurons PDE10A inhibitors by up-regulating cAMP and cGMP levels act as D1 agonists and D2 antagonists because the activation of Gs-protein coupled dopamine D1 receptor increases intracellular cAMP, whereas the activation of the Gi-protein coupled dopamine D2 receptor decreases intracellular cAMP levels through inhibition of adenylyl cyclase activity (Mutschler et al., 2001).

Elevated intracellular cAMP levels mediated by D1 receptor signalling seems to modulate a series of neuronal processes responsible for working memory in the prefrontal cortex (Sawaguchi, 2000), and it is reported that D1 receptor activation may improve working memory deficits in schizophrenic patients (Castner et al., 2000). Thus, it seems likely that a further enhancement of this pathway might also improve the cognitive symptoms of schizophrenia.

Further indication of an effect of PDE10A inhibition on negative symptoms of psychosis are given by Rodefer et al. (2005) who could show that papaverine reverses attentional set-shifting deficits induced by subchronic administration of phencyclidine, an NMDA antagonist, in rats. Attentional deficits including an impairment of shifting attention to novel stimuli belongs to the negative symptoms of schizophrenia. In the study the attentional deficits were induced by administering phencyclidine for 7 days followed by a washout period. The PDE10A inhibitor papaverine was able to reverse the enduring deficits induced by the subchronic treatment.

Imidazo[1,5-a]pyrido[3,2-e]pyrazinones its synthesis and some medical uses are well described in patents and the literature.

The applications EP 0 400 583 and U.S. Pat. No. 5,055,465 from Berlex Laboratories, Inc. disclose a group of imidazoquinoxalinones, their aza analogs and a process for their preparation. These compounds have been found to have inodilatory, vasodilatory and venodilatory effects. The therapeutic activity is based on the inhibition of phosphodiesterase 3 (PDE3).

EP 0 736 532 discloses pyrido[3,2-e]pyrazinones and a process for their preparation. These compounds are described to have anti-asthmatic and anti-allergic properties. Examples of this invention are inhibitors of PDE4 and PDE5.

WO 00/43392 discloses the use of imidazo[1,5-a]pyrido[3,2-e]pyrazinones which are inhibitors of PDE3 and PDE5 for the therapy of erectile dysfunction, heart failure, pulmonic hypertonia and vascular diseases which are accompanied by insufficient blood supply.

An other group of pyrido[3,2-e]pyrazinones, disclosed in WO 01/68097 are inhibitors of PDE5 and can be used for the treatment of erectile dysfunction.

Further methodes for the preparation of imidazo[1,5-a]pyrido[3,2-e]pyrazinones are described also by D. Norris et al. (Tetrahedron Letters 42 (2001), 4297-4299).

WO 92/22552 refers to imidazo[1,5-a]quinoxalines which are generally substituted at position 3 with a carboxylic acid group and derivatives thereof. These compounds are described to be useful as anxiolytic and sedative/hypnotic agents.

In contrast only a limited number of imidazo[1,5-a]pyrido[3,2-e]pyrazines and their medical use are already published.

WO 99/45009 refers to a group of imidazopyrazines of formula (I)

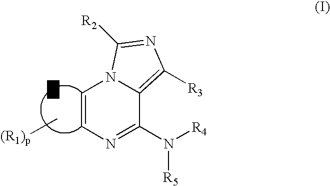

Part of the definition of Q is to form a 6-membered heterocyclic ring including pyridine. While R₁, R₂ and R₃ are representing a large variety of substituents, the definition of the group —NR₄R₅ is of special importance.

R₄ and R₅ are each independently hydrogen, R₆ or —C(O)R₆ or the whole group NR₄R₅ forms a 3- to 8-membered saturated or unsaturated ring. R₆ is alkyl, alkenyl, alkynyl, cycloalkyl, cycloalkylalkyl, cycloalkenyl, cycloalkenylalkyl, aryl, aralkyl, heterocyclo or heterocycloalkyl, each of which is unsubstituted or substituted.

The compounds are described to be inhibitors of protein tyrosine kinases used in the treatment of protein tyrosine kinase-associated disorders such as immunologic disorders.

Interestingly, for all examples listed in claim 9 the structure of the group NR₄R₅ is limited in a way that one of R₄ and R₅ is hydrogen and for the other one R₆ is phenyl (unsubstituted or substituted).

This structural selection of the group NR₄R₅ is inline with published SAR data from the same company (P. Chen et al., Bioorg. Med. Chem. Lett. 12 (2002), 1361-1364 and P. Chen et al., Bioorg. Med. Chem. Lett. 12 (2002), 3153-3156).

### SUMMARY OF THE INVENTION

This invention relates to compounds of formula (II) and to pharmaceutically acceptable salts, solvates and prodrugs thereof.

Compounds of formula (II) 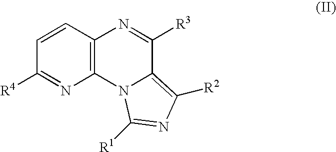
wherein R¹ and R² are independently selected from
H,
a cyclic radical,
C₁₋₈ alkyl or C₃₋₈ cycloalkyl, optionally mono- or polysubstituted with halo, OH, O—C₁₋₃ alkyl, and/or a cyclic radical,
C₂₋₈ alkenyl or C₃₋₈ cycloalkenyl, optionally mono- or polysubstituted with halo, OH, O—C₁₋₃ alkyl and/or a cyclic radical,
C₂-C₈ alkynyl, optionally mono- or polysubstituted with halo, OH, O—C₁₋₃-alkyl, and/or a cyclic radical,
a saturated, monounsaturated or polyunsaturated heterocycle with 5 to 15 ring atoms, optionally mono- or polysubstituted with halo, amino, C₁₋₃ alkylamino, di-C₁₋₃ alkylamino, nitro, C₁₋₃ alkyl, and/or O—C₁₋₃ alkyl, and phenyl, optionally mono- or polysubstituted with halo, amino, C₁₋₃ alkylamino, di-C₁₋₃ alkylamino, nitro, C₁₋₃ alkyl, and/or OC₁₋₃ alkyl and/or a cyclic radical,
R³ is NH₂, NHR⁵ or NR⁵R⁶;
wherein R⁵ and R⁶ are independently selected from
a cyclic radical,
C₁₋₅ alkyl, optionally mono- or polysubstituted with halo, OH, O—C₁₋₃ alkyl and/or a cyclic radical,
aryl-C₁₋₅-alkyl wherein aryl is phenyl, optionally mono- or polysubstituted with halo, nitro, C₁₋₃ alkyl, OC₁₋₃ alkyl, and/or a cyclic radical,
(C═O)—C₁₋₅ alkyl optionally mono- or polysubstituted with halo, OH, O—C₁₋₃ alkyl and/or a cyclic radical,
NR⁵R⁶ together form a saturated or unsaturated five-, six- or seven-membered ring which can contain up to 3 heteroatoms, preferably N including N-oxide, S and O, optionally mono- or polysubstituted with halo, C₁₋₃ alkyl, O—C₁₋₃ alkyl and/or aryl-C₁₋₅-alkyl, wherein aryl is phenyl, optionally mono- or polysubstituted with halo, nitro, C₁₋₃ alkyl, and/or O—C₁₋₃ alkyl, and/or a cyclic radical, and
R⁴ is selected from
H,
halo,
a cyclic radical,
R⁷,
OH or OR⁷,
NH(C═O)—C₁₋₃ alkyl, optionally mono- or polysubstituted with halo, OH, O—C₁₋₃ alkyl and/or a cyclic radical, in particular aryl or phenyl, or
NH₂, NHR⁷ or NR⁷R⁸,
wherein R⁷ and R⁸ are independently selected from
a cyclic radical,
C₁₋₆ alkyl or C₃₋₆ cycloalkyl, optionally mono- or polysubstituted with halo, OH, O—C₁₋₃ alkyl, and/or a cyclic radical,
aryl-C₁₋₅-alkyl wherein aryl is phenyl, optionally mono- or polysubstituted with halo, nitro, C₁₋₃ alkyl, O—C₁₋₃ alkyl, and/or a cyclic radical,
NR⁷R⁸ together form a saturated or unsaturated five- or six-membered ring which can contain up to 3 heteroatoms, preferably N including N-oxide, S and O, optionally mono- or polysubstituted with halo, C₁₋₃ alkyl, C₃₋₆ cycloalkyl, O—C₁₋₃ alkyl and/or aryl-C₁₋₅-alkyl, wherein aryl is phenyl, optionally mono- or polysubstituted with halo, amino, C₁₋₃ alkylamino, di-C₁₋₃ alkylamino, nitro, C₁₋₃ alkyl, O—C₁₋₃ alkyl and/or a cyclic radical,
or pharmaceutically acceptable salts and derivatives thereof.

### BRIEF DESCRIPTION OF THE FIGURES

FIG. 1 shows silver staining and a Western blot showing detection of PDE10.

FIG. 2 shows that the main port of PDE10 was found in the membrane fraction.

FIG. 3 shows the gene alignment of the PDE10 rat cat domain, PD10 guinea pig P4-P3, PDE10 pig P1-P2 and consensus.

FIG. 4 shows differences in the gene alignment of the catalytic domain of genes shown in FIG. 3

FIG. 5 shows graphs of the activity and stereotyped sniffing score after administration of the compounds of Examples 1 and 11 compared to controls with and without MK-801 stimulation.

### DETAILED DESCRIPTION

The term “halo” refers to fluoro, chloro, bromo or iodo.

The terms “alkyl”, alkenyl” and “alkynyl” refer to straight or branched radicals with up to 8 carbon atoms preferably up to 6 carbon atoms and more preferably up to 5 carbon atoms such as methyl, ethyl, vinyl, ethynyl, propyl, allyl, propynyl, butyl, butenyl, butynyl etcl. which may optionally be substituted as indicated above.

The term “cyclic radical” refers to saturated, unsaturated or aromatic carbocyles or carboheterocycles, optionally mono- or polysubstituted with halo, amino, C₁₋₃ alkylamino, di-C₁₋₃ alkylamino, nitro, C₁₋₃ alkyl, OH, O—C₁₋₃ alkyl and/or a cyclic radical. The cyclic radical preferably contains 3 to 20, in particular 4 to 10 C-atoms. Carboheterocyles may contain 1 to 6, in particular 1 to 3 heteroatoms, preferably selected from O, N, S and/or P. The cyclic radical can be bound via a C-atom or optionally via a N, O, S, SO or SO₂-group. An example for a cyclic radical is phenyl.

A preferred embodiment of this invention relates to compounds of formula (II) wherein R¹ is selected from

H,

C₁₋₄ alkyl, particularly C₂₋₄ alkyl optionally mono- or polysubstituted with halo, OH, C₁₋₃ alkyl, or/and a cyclic radical or

phenyl, optionally mono- or polysubstituted with halo, amino, C₁₋₃ alkylamino, di-C₁₋₃ alkylamino, nitro, C₁₋₃ alkyl, O—C₁₋₃ alkyl or/and a cyclic radical. Especially preferred are C₂₋₄-alkyl or phenyl.

An other preferred embodiment of this invention relates to compounds of formula (II) wherein R² is

H or

C₁₋₄ alkyl optionally halogenated, particularly methyl or trifluoromethyl. Especially preferred are hydrogen or a methyl-group.

A further preferred embodiment of this invention relates to compounds of formula (II) wherein R³ is selected from

NH₂,

NHC₁₋₃ alkyl, optionally mono- or polysubstituted with halo, OH, O—C₁₋₃ alkyl and/or a cyclic radical, or

NH(C═O)—C₁₋₃ alkyl, optionally mono- or polysubstituted with halo, OH, O—C₁₋₃ alkyl and/or a cyclic radical or

cyclopropyl, cyclobutyl, tetrahydropyrrolyl, pyrrolyl, pyrazolyl, imidazolyl, 1,2,3-triazolyl, 1,2,4-triazolyl, piperidinyl, morpholinyl, piperazinyl, optionally substituted with C₁₋₃ alkyl, optionally mono- or polysubstituted with halo, OH and/or O—C₁₋₃ alkyl, or arylalkyl, wherein aryl is phenyl, optionally mono- or polysubstituted with halo, amino, C₁₋₃ alkylamino, di-C₁₋₃ alkylamino, nitro, C₁₋₃ alkyl, and/or O—C₁₋₃ alkyl and/or a cyclic radical, for example

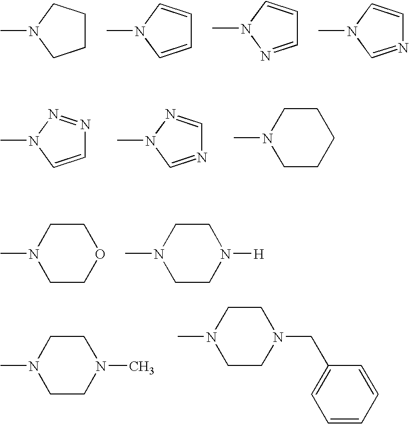

Especially preferred is one of —NH₂, —NH—C₁₋₃-alkyl, —NH—(C═O)—C₁₋₃-alkyl or -imidazolyl.

Also a preferred embodiment of this invention relates to compounds of formula (II) wherein R⁴ is selected from

OH or O—C₁₋₃ alkyl, optionally mono- or polysubstituted with halo, OH, O—C₁₋₃ alkyl, and/or a cyclic radical,

NHC₁₋₃ alkyl, optionally mono- or polysubstituted with halo, OH, O—C₁₋₃ alkyl and/or a cyclic radical, or

NH benzyl, wherein the phenyl group is phenyl, optionally mono- or polysubstituted with halo, amino, C₁₋₃ alkylamino, di-C₁₋₃ alkylamino, nitro, C₁₋₃ alkyl, O—C₁₋₃ alkyl and/or a cyclic radical or

cyclopropyl, cyclobutyl, tetrahydropyrrolyl, pyrrolyl, pyrazolyl, imidazolyl, 1,2,3-triazolyl, 1,2,4-triazolyl, piperidinyl, morpholinyl, piperazinyl, optionally substituted with C₁₋₃ alkyl, optionally mono- or polysubstituted with halo, OH, C₁₋₅ alkyl and/or O—C₁₋₃ alkyl, or arylalkyl, wherein aryl is phenyl, optionally mono- or polysubstituted with halo, amino, C₁₋₃ alkylamino, di-C₁₋₃ alkylamino, nitro, C₁₋₃ alkyl, O—C₁₋₃ alkyl and/or a cyclic radical.

Especially preferred are one of hydrogen, —O—C₁₋₃-alkyl, —NH—C₁₋₃-alkyl, —NH-benzyl or the following groups:

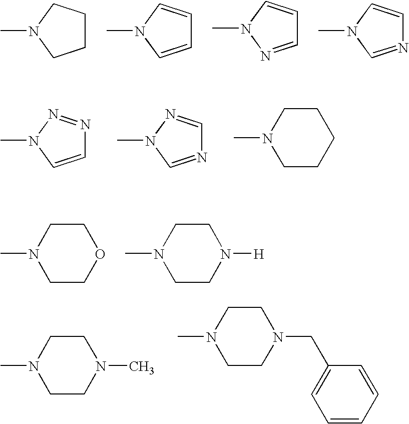

The compounds of formula (II) are inhibitors of phosphodiesterase 10 and thus have new biological properties. Based on these properties therapeutic uses of compounds of formula (II) which are different from those disclosed in WO 99/45009 are part of this invention.

Examples of specific compounds of the formula (II) are the following:
4-amino-8-methoxy-3-methyl-1-propyl-imidazo[1,5-a]pyrido[3,2-e]pyrazine
4-amino-1-ethyl-8-methoxy-3-methyl-imidazo[1,5-a]pyrido[3,2-e]pyrazine
4-amino-1-ethyl-3-methyl-imidazo[1,5-a]pyrido[3,2-e]pyrazine
4-amino-3-methyl-1-propyl-imidazo[1,5-a]pyrido[3,2-e]pyrazine
4-amino-1-ethyl-8-(2-ethyl-4-methyl-imidazol-1-yl)-3-methyl-imidazo[1,5-a]pyrido[3,2-e]pyrazine
4-amino-3-methyl-1-propyll-8-(2-propyl-4-methyl-imidazol-1-yl)-imidazo[1,5-a]pyrido[3,2-e]pyrazine
4-amino-1-hexyl-8-methoxy-3-methyl-imidazo[1,5-a]pyrido[3,2-e]pyrazine
4-amino-8-methoxy-3-methyl-1-(3,3,3-trifluoropropyl)-imidazo[1,5-a]pyrido[3,2-e]pyrazine
4-amino-8-methoxy-3-methyl-1-phenethyl-imidazo[1,5-a]pyrido[3,2-e]pyrazine
4-amino-8-methoxy-3-methyl-1-phenyl-imidazo[1,5-a]pyrido[3,2-e]pyrazine
4-amino-1-(2-chloro-phenyl)-8-methoxy-3-methyl-imidazo[1,5-a]pyrido[3,2-e]pyrazine
4-amino-1-(4-fluoro-phenyl)-8-methoxy-3-methyl-imidazo[1,5-a]pyrido[3,2-e]pyrazine
4-amino-1-isopropyl-8-methoxy-imidazo[1,5-a]pyrido[3,2-e]pyrazine
4-amino-8-methoxy-imidazo[1,5-a]pyrido[3,2-e]pyrazine
4-amino-8-methoxy-3-phenyl-imidazo[1,5-a]pyrido[3,2-e]pyrazine
4-(N-methyl-amino)-8-methoxy-3-methyl-1-propyl-imidazo[1,5-a]pyrido[3,2-e]pyrazine
4-(N-ethyl-amino)-8-methoxy-3-methyl-1-propyl-imidazo[1,5-a]pyrido[3,2-e]pyrazine
4-(N-methyl-amino)-1-ethyl-8-methoxy-3-methyl-imidazo[1,5-a]pyrido[3,2-e]pyrazine
4-(N,N-dimethyl-amino)-8-methoxy-3-methyl-1-propyl-imidazo[1,5-a]pyrido[3,2-e]pyrazine
4-(N-butyl-amino)-1-ethyl-8-methoxy-3-methyl-imidazo[1,5-a]pyrido[3,2-e]pyrazine
4-(N-benzyl-amino)-1-ethyl-8-methoxy-3-methyl-imidazo[1,5-a]pyrido[3,2-e]pyrazine
4-(N-cyclopentyl-amino)-1-ethyl-8-methoxy-3-methyl-imidazo[1,5-a]pyrido[3,2-e]pyrazine
4-(N-cyclopentyl-amino)-8-methoxy-3-methyl-1-propyl-imidazo[1,5-a]pyrido[3,2-e]pyrazine
1-ethyl-8-methoxy-3-methyl-4-morpholino-imidazo[1,5-a]pyrido[3,2-e]pyrazine
4-azetidine-8-methoxy-3-methyl-1-(3,3,3-trifluoropropyl)-imidazo[1,5-a]pyrido[3,2-e]pyrazine
8-methoxy-3-methyl-1-propyl-4-pyrrolidino-imidazo[1,5-a]pyrido[3,2-e]pyrazine
8-methoxy-3-methyl-4-piperidino-1-propyl-imidazo[1,5-a]pyrido[3,2-e]pyrazine
1-ethyl-8-methoxy-3-methyl-4-(4-phenylpiperazino)-imidazo[1,5-a]pyrido[3,2-e]pyrazine
8-methoxy-3-methyl-1-propyl-4-(pyrazol-1-yl)-imidazo[1,5-a]pyrido[3,2-e]pyrazine
8-methoxy-3-methyl-1-propyl-4-(pyrazol-1-yl)-imidazo[1,5-a]pyrido[3,2-e]pyrazine hydro chloride
4-(imidazol-1-yl)-8-methoxy-3-methyl-1-propyl-imidazo[1,5-a]pyrido[3,2-e]pyrazine
8-methoxy-3-methyl-1-propyl-4-(1,2,3-triazol-1-yl)-imidazo[1,5-a]pyrido[3,2-e]pyrazine
8-methoxy-3-methyl-1-propyl-4-(1,2,4-triazol-1-yl)-imidazo[1,5-a]pyrido[3,2-e]pyrazine
8-methoxy-3-methyl-4-(2-methyl-imidazol-1-yl)-1-propyl-imidazo[1,5-a]pyrido[3,2-e]pyrazine
4-(imidazol-1-yl)-3-methyl-1-propyl-imidazo[1,5-a]pyrido[3,2-e]pyrazine-8-ol
1-ethyl-4-(N-formyl-amino)-8-methoxy-3-methyl-imidazo[1,5-a]pyrido[3,2-e]pyrazine
4-(N-formyl-amino)-8-methoxy-3-methyl-1-propyl-imidazo[1,5-a]pyrido[3,2-e]pyrazine
4-(N-acetyl-amino)-8-methoxy-3-methyl-1-propyl-imidazo[1,5-a]pyrido[3,2-e]pyrazine
4-(N,N-diacetyl-amino)-8-methoxy-3-methyl-1-propyl-imidazo[1,5-a]pyrido[3,2-e]pyrazine
4-(N-acetyl-amino)-1-ethyl-8-methoxy-3-methyl-imidazo[1,5-a]pyrido[3,2-e]pyrazine
4-(N,N-diacetyl-amino)-1-ethyl-8-methoxy-3-methyl-imidazo[1,5-a]pyrido[3,2-e]pyrazine
4-(N-acetyl-amino)-8-methoxy-3-methyl-1-phenyl-imidazo[1,5-a]pyrido[3,2-e]pyrazine
8-methoxy-3-methyl-4-(N-propionyl-amino)-1-propyl-imidazo[1,5-a]pyrido[3,2-e]pyrazine
4-(N-cyclopropylcarboxy-amino)-8-methoxy-3-methyl-1-propyl-imidazo[1,5-a]pyrido[3,2-e]pyrazine
   and pharmaceutically acceptable salts and derivatives thereof.

Further, it was found that compounds of formula (IV)

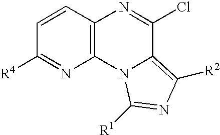

wherein X is Cl or Br and R¹, R² and R⁴ are as defined above are potent inhibitors of phosphodiesterase 10.

The above listed compounds and their pharmaceutically salts, solvates, and prodrugs thereof are preferred embodiments of the subject invention.

The invention furthermore relates to the physiologically acceptable salts, solvates and derivatives of the compounds according to formula (II) or (IV). Derivatives of the compounds according to formula (II) or (IV) are, for example, amides, esters and ethers. Further, the term “derivative” also encompasses prodrugs and metabolites of compounds of formula (II) or (IV).

The physiologically acceptable salts may be obtained by neutralizing the bases with inorganic or organic acids or by neutralizing the acids with inorganic or organic bases. Examples of suitable inorganic acids are hydrochloric acid, sulphuric acid, phosphoric acid or hydrobromic acid, while examples of suitable organic acids are carboxylic acid, sulpho acid or sulphonic acid, such as acetic acid, tartaric acid, lactic acid, propionic acid, glycolic acid, malonic acid, maleic acid, fumaric acid, tannic acid, succinic acid, alginic acid, benzoic acid, 2-phenoxybenzoic acid, 2-acetoxybenzoic acid, cinnamic acid, mandelic acid, citric acid, maleic acid, salicylic acid, 3-aminosalicylic acid, ascorbic acid, embonic acid, nicotinic acid, isonicotinic acid, oxalic acid, gluconic acid, amino acids, methanesulphonic acid, ethanesulphonic acid, 2-hydroxyethanesulphonic acid, ethane-1,2-disulphonic acid, benzenesulphonic acid, 4-methylbenzenesulphonic acid or naphthalene-2-sulphonic acid. Examples of suitable inorganic bases are sodium hydroxide, potassium hydroxide and ammonia, while examples of suitable organic bases are amines, preferably, however, tertiary amines, such as trimethylamine, triethylamine, pyridine, N,N-dimethylaniline, quinoline, isoquinoline, α-picoline, β-picoline, γ-picoline, quinaldine and pyrimidine.

In addition, physiologically acceptable salts of the compounds according to formula (II) or (IV) can be obtained by converting derivatives which possess tertiary amino groups into the corresponding quaternary ammonium salts in a manner known per se using quaternizing agents. Examples of suitable quaternizing agents are alkyl halides, such as methyl iodide, ethyl bromide and n-propyl chloride, and also arylalkyl halides, such as benzyl chloride or 2-phenylethyl bromide.

Furthermore, in the case of the compounds of the formula (II) or (IV) which contain an asymmetric carbon atom, the invention relates to the D form, the L form and D,L mixtures and also, where more than one asymmetric carbon atom is present, to the diastereomeric forms. Those compounds of the formula (II) or (IV) which contain asymmetric carbon atoms, and which as a rule accrue as racemates, can be separated into the optically active isomers in a known manner, for example using an optically active acid. However, it is also possible to use an optically active starting substance from the outset, with a corresponding optically active or diastereomeric compound then being obtained as the end product.

The compounds according to the invention have been found to have pharmacologically important properties which can be used therapeutically. The compounds according to formula (II) or (IV) can be used alone, in combination with each other or in combination with other active compounds. The compounds according to the invention are inhibitors of phosphodiesterase 10. It is therefore a part of the subject-matter of this invention that the compounds according to formula (II) or (IV), and their salts and also pharmaceutical preparations which comprise these compounds or their salts, can be used for treating or preventing disorders caused by, associated with and/or accompanied by phosphodiesterase 10 hyperactivity and/or disorders in which inhibiting phosphodiesterase 10 is of value.

It is an embodiment of this invention, that compounds of formula (II) or (IV) including their salts, solvates and prodrugs and also pharmaceutical compositions comprising an amount of a compound of formula (II) or (IV) or one of its salts, solvates or prodrugs effective in inhibiting PDE10 can be used for the treatment of central nervous system disorders of mammals including a human.

More particularly, the invention relates to the treatment of neurological and psychiatric disorders including, but not limited to, (1) schizophrenia and other psychotic disorders; (2) mood [affective] disorders; (3) neurotic, stress-related and somatoform disorders including anxiety disorders; (4) eating disorders; sexual dysfunction comprising excessive sexual drive; (5) disorders of adult personality and behaviour; (6) disorders usually first diagnosed in infancy, childhood and adolescence; (7) mental retardation and (8) disorders of psychological development; (9) disorders comprising the symptom of cognitive deficiency in a mammal, including a human; (10) factitious disorders.

(1) Examples of schizophrenia and other psychotic disorders disorders that can be treated according to the present invention include, but are not limited to, continuous or episodic schizophrenia of different types (for instance paranoid, hebephrenic, catatonic, undifferentiated, residual, and schizophreniform disorders); schizotypal disorders (such as borderline, latent, prepsychotic, prodromal, pseudoneurotic pseudopsychopathic schizophrenia and schizotypal personality disorder); persistent delusional disorders; acute, transient and persistent psychotic disorders; induced delusional disorders; schizoaffective disorders of different type (for instance manic, depressive or mixed type); puerperal psychosis and other and unspecified nonorganic psychosis.

(2) Examples of mood [affective] disorders that can be treated according to the present invention include, but are not limited to, manic episodes associated to bipolar disorder and single manic episodes, hypomania, mania with psychotic symptoms; bipolar affective disorders (including for instance bipolar affective disorders with current hypomanic and manic episodes with or without psychotic symptoms); depressive disorders, such as single episode or recurrent major depressive disorder, depressive disorder with postpartum onset, depressive disorders with psychotic symptoms; persistent mood [affective] disorders, such as cyclothymia, dysthymia; premenstrual dysphoric disorder.

(3) Examples of disorders belonging to the neurotic, stress-related and somatoform disorders that can be treated according to the present invention include, but are not limited to, phobic anxiety disorders, for instance agoraphobia and social phobia primarily but not exclusively related to psychosis; other anxiety disorders such as panic disorders and general anxiety disorders; obsessive compulsive disorder; reaction to sever stress and adjustment disorders, such as post traumatic stress disorder; dissociative disorders and other neurotic disorders such as depersonalisation-derealisation syndrome.

(5) Examples of disorders of adult personality and behaviour that can be treated according to the present invention include, but are not limited to, specific personality disorders of the paranoid, schizoid, schizotypal, antisocial, borderline, histrionic, narcissistic, avoidant, dissocial, emotionally unstable, anankastic, anxious and dependent type; mixed personality disorders; habit and impulse disorders (such as trichotillomania, pyromania, maladaptive aggression); disorders of sexual preference.

(6) Examples of disorders usually first diagnosed in infancy, childhood and adolescence that can be treated according to the present invention include, but are not limited to, hyperkinetic disorders, attentional deficit/hyperactivity disorder (AD/HD), conduct disorders; mixed disorders of conduct and emotional disorders; nonorganic enuresis, nonorganic encopresis; stereotyped movement disorder; and other specified behavioural emotional disorders, such as attention deficit disorder without hyperactivity, excessive masturbation, nail-biting, nose-picking and thumb-sucking; disorders of psychological development particularly schizoid disorder of childhood and pervasive development disorders such as psychotic episodes associated to Asperger's syndrome.

(8) Examples of disorders of psychological development include but are not limited to developmental disorders of speech and language, developmental disorders of scholastic skills, such as specific disorder of arithmetical skills, reading disorders and spelling disorders and other learning disorders. These disorders are predominantly diagnosed in infancy, childhood and adolescencs.

(9) The phrase “cognitive deficiency” as used here in “disorder comprising as a symptom cognitive deficiency” refers to a subnormal functioning or a suboptimal functioning in one or more cognitive aspects such as memory, intellect, learning and logic ability, or attention in a particular individual comparative to other individuals within the same general age population.

(10) Examples of disorders comprising as a symptom cognitive deficiency that can be treated according to the present invention include, but are not limited to, cognitive deficits primarily but not exclusively related to psychosis; age-associated memory impairment, Parkinson's disease, Alzheimer's disease, multi infarct dementia, Lewis body dementia, stroke, frontotemporal dementia, progressive supranuclear palsy Huntington's disease and in HIV disease, cerebral trauma and drug abuse; mild cognitive disorder.

(11) Additionally, the invention relates to movement disorders with malfunction of basal ganglia. Examples of movement disorders with malfunction of basal ganglia that can be treated according to the present invention include, but are not limited to, different subtypes of dystonia, such as focal dystonias, multiple-focal or segmental dystonias, torsion dystonia, hemispheric, generalised and tardive dyskinesias (induced by psychopharmacological drugs), akathisias, dyskinesias such as Huntington's disease, Parkinson's disease, Lewis body disease, restless leg syndrome, PLMS.

(12) Furthermore the invention relates to the treatment of organic, including symptomatic mental disorders, especially to organic delusional (schizophrenia-like) disorders, presenil or senile psychosis associated to dementia, to psychosis in epilepsy and Parkinson's disease and other organic and symptomatic psychosis; delirium; infective psychosis; personality and behavioural disorders due to brain disease, damage and dysfunction.

(13) The invention relates to the treatment of mental and behavioural disorders due to psychoactive compounds, more particular to the treatment of psychotic disorders and residual and late-onset psychotic disorders induced by alcohol, opioids, cannabinoids, cocaine, hallucinogens, other stimulants, including caffeine, volatile solvents and other psychoactive compounds.

(14) The invention further relates to a general improvement of learning and memory capacities in a mammal, including a human.

An effective dose of the compounds according to the invention, or their salts, is used, in addition to physiologically acceptable carriers, diluents and/or adjuvants for producing a pharmaceutical composition. The dose of the active compounds can vary depending on the route of administration, the age and weight of the patient, the nature and severity of the diseases to be treated, and similar factors. The daily dose can be given as a single dose, which is to be administered once, or be subdivided into two or more daily doses, and is as a rule 0.001-2000 mg. Particular preference is given to administering daily doses of 0.1-500 mg, e.g. 0.1-100 mg.

Suitable administration forms are oral, parenteral, intravenous, transdermal, topical, inhalative, intranasal and sublingual preparations. Particular preference is given to using oral, parenteral, e.g. intravenous or intramuscular, intranasal, e.g. dry powder or sublingual preparations of the compounds according to the invention. The customary galenic preparation forms, such as tablets, sugar-coated tablets, capsules, dispersible powders, granulates, aqueous solutions, alcohol-containing aqueous solutions, aqueous or oily suspensions, syrups, juices or drops, are used.

Solid medicinal forms can comprise inert components and carrier substances, such as calcium carbonate, calcium phosphate, sodium phosphate, lactose, starch, mannitol, alginates, gelatine, guar gum, magnesium stearate, aluminium stearate, methyl cellulose, talc, highly dispersed silicic acids, silicone oil, higher molecular weight fatty acids, (such as stearic acid), gelatine, agar agar or vegetable or animal fats and oils, or solid high molecular weight polymers (such as polyethylene glycol); preparations which are suitable for oral administration can comprise additional flavourings and/or sweetening agents, if desired.

Liquid medicinal forms can be sterilized and/or, where appropriate, comprise auxiliary substances, such as preservatives, stabilizers, wetting agents, penetrating agents, emulsifiers, spreading agents, solubilizers, salts, sugars or sugar alcohols for regulating the osmotic pressure or for buffering, and/or viscosity regulators.

Examples of such additives are tartrate and citrate buffers, ethanol and sequestering agents (such as ethylenediaminetetraacetic acid and its non-toxic salts). High molecular weight polymers, such as liquid polyethylene oxides, microcrystalline celluloses, carboxymethyl celluloses, polyvinylpyrrolidones, dextrans or gelatine, are suitable for regulating the viscosity. Examples of solid carrier substances are starch, lactose, mannitol, methyl cellulose, talc, highly dispersed silicic acids, high molecular weight fatty acids (such as stearic acid), gelatine, agar agar, calcium phosphate, magnesium stearate, animal and vegetable fats, and solid high molecular weight polymers, such as polyethylene glycol.

Oily suspensions for parenteral or topical applications can be vegetable synthetic or semisynthetic oils, such as liquid fatty acid esters having in each case from 8 to 22 C atoms in the fatty acid chains, for example palmitic acid, lauric acid, tridecanoic acid, margaric acid, stearic acid, arachidic acid, myristic acid, behenic acid, pentadecanoic acid, linoleic acid, elaidic acid, brasidic acid, erucic acid or oleic acid, which are esterified with monohydric to trihydric alcohols having from 1 to 6 C atoms, such as methanol, ethanol, propanol, butanol, pentanol or their isomers, glycol or glycerol. Examples of such fatty acid esters are commercially available miglyols, isopropyl myristate, isopropyl palmitate, isopropyl stearate, PEG 6-capric acid, caprylic/capric acid esters of saturated fatty alcohols, polyoxyethylene glycerol trioleates, ethyl oleate, waxy fatty acid esters, such as artificial ducktail gland fat, coconut fatty acid isopropyl ester, oleyl oleate, decyl oleate, ethyl lactate, dibutyl phthalate, diisopropyl adipate, polyol fatty acid esters, inter alia. Silicone oils of differing viscosity, or fatty alcohols, such as isotridecyl alcohol, 2-octyldodecanol, cetylstearyl alcohol or oleyl alcohol, or fatty acids, such as oleic acid, are also suitable. It is furthermore possible to use vegetable oils, such as castor oil, almond oil, olive oil, sesame oil, cotton seed oil, groundnut oil or soybean oil.

Suitable solvents, gelatinizing agents and solubilizers are water or water-miscible solvents. Examples of suitable substances are alcohols, such as ethanol or isopropyl alcohol, benzyl alcohol, 2-octyldodecanol, polyethylene glycols, phthalates, adipates, propylene glycol, glycerol, di- or tripropylene glycol, waxes, methyl cellosolve, cellosolve, esters, morpholines, dioxane, dimethyl sulphoxide, dimethylformamide, tetrahydrofuran, cyclohexanone, etc.

Cellulose ethers which can dissolve or swell both in water or in organic solvents, such as hydroxypropylmethyl cellulose, methyl cellulose or ethyl cellulose, or soluble starches, can be used as film-forming agents.

Mixtures of gelatinizing agents and film-forming agents are also perfectly possible. In this case, use is made, in particular, of ionic macromolecules such as sodium carboxymethyl cellulose, polyacrylic acid, polymethacrylic acid and their salts, sodium amylopectin semiglycolate, alginic acid or propylene glycol alginate as the sodium salt, gum arabic, xanthan gum, guar gum or carrageenan. The following can be used as additional formulation aids: glycerol, paraffin of differing viscosity, triethanolamine, collagen, allantoin and novantisolic acid. Use of surfactants, emulsifiers or wetting agents, for example of Na lauryl sulphate, fatty alcohol ether sulphates, di-Na—N-lauryl-β-iminodipropionate, polyethoxylated castor oil or sorbitan monooleate, sorbitan monostearate, polysorbates (e.g. Tween), cetyl alcohol, lecithin, glycerol monostearate, polyoxyethylene stearate, alkylphenol polyglycol ethers, cetyltrimethylammonium chloride or mono-/dialkylpolyglycol ether orthophosphoric acid monoethanolamine salts can also be required for the formulation. Stabilizers, such as montmorillonites or colloidal silicic acids, for stabilizing emulsions or preventing the breakdown of active substances such as antioxidants, for example tocopherols or butylhydroxyanisole, or preservatives, such as p-hydroxybenzoic acid esters, can likewise be used for preparing the desired formulations.

Preparations for parenteral administration can be present in separate dose unit forms, such as ampoules or vials. Use is preferably made of solutions of the active compound, preferably aqueous solution and, in particular, isotonic solutions and also suspensions. These injection forms can be made available as ready-to-use preparations or only be prepared directly before use, by mixing the active compound, for example the lyophilisate, where appropriate containing other solid carrier substances, with the desired solvent or suspending agent.

Intranasal preparations can be present as aqueous or oily solutions or as aqueous or oily suspensions. They can also be present as lyophilisates which are prepared before use using the suitable solvent or suspending agent.

Inhalable preparations can present as powders, solutions or suspensions. Preferably, inhalable preparations are in the form of powders, e.g. as a mixture of the active ingredient with a suitable formulation aid such as lactose.

The preparations are produced, aliquoted and sealed under the customary antimicrobial and aseptic conditions.

As indicated above, the compounds of the invention may be administered as a combination therapy with further active agents, e.g. therapeutically active compounds useful in the treatment of central nervous system disorders. These further compounds may be PDE10 inhibitors or compounds which have an activity which is not based on PDE10 inhibition such as dopamine D2 receptor modulating agents or NMDA modulating agents.

For a combination therapy, the active ingredients may be formulated as compositions containing several active ingredients in a single dose form and/or as kits containing individual active ingredients in separate dose forms. The active ingredients used in combination therapy may be co-administered or administered separately.

The invention furthermore relates to processes for preparing the compounds according to the invention.

The synthesis of compounds of formula (II) starts from imidazo[1,5-a]pyrido[3,2-e]pyrazinones of formula (III),

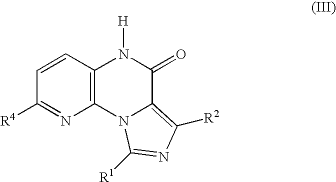

wherein R¹, R² and R⁴ are as described above.

The preparation of compounds of formula (III) is well described e.g. in WO 00/43392, WO 01/68097 and also by D. Norris et al. (Tetrahedron Letters 42 (2001), 4297-4299).

According to standard procedures known from the literature and already used in WO 99/45009 compounds of formula (III) are halogenated by treatment with halogenating reagents like POCl₃, PCl₃, PCl₅, SOCl₂, POBr₃, PBr₃ or PBr₅, yielding e.g. 4-chloro or 4-bromo-imidazo[1,5-a]pyrido[3,2-e]pyrazines of formula (IV),

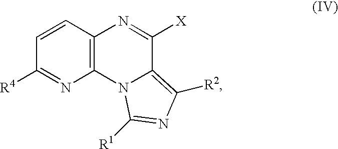

wherein X is Cl or Br, particularly Cl, and R¹, R² and R⁴ are as defined above.

Following this the chloro or bromo atom is substituted by amine treatment forming compounds of formula (II). Compounds of formula (II) with R⁵ and or R⁶ representing hydrogen can be transformed into N-acylated derivatives by the reaction with a very reactive carboxylic acid derivative. Carboxylic acid chlorides and anhydride are used preferentially.

### EXAMPLES

### Intermediate A1

### 4-chloro-8-methoxy-3-methyl-1-propyl-imidazo[1,5-a]pyrido[3,2-e]pyrazine

16 g of 8-methoxy-3-methyl-1-propyl-imidazo[1,5-a]pyrido[3,2-e]pyrazine-4-one and 120 ml POCl₃ are mixed and heated up to reflux for 8 hours. After cooling to room temperature the reaction mixture is treated with 1200 ml crushed ice/water and stirred for 1 hour. The product is extracted with 2×300 ml dichloromethane. The collected organic layer is washed with 2×300 ml water and dried with Na₂SO₄. The solvent is removed under reduced pressure.

Yield: 14.5 g

m.p.: 121-123° C.

Many other intermediates A of formula (IV) can be prepared according to this procedure. Some examples are the following:

| | |
|---|---|
| | (IV) |
| 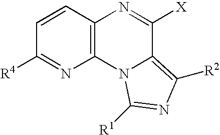 | |

| Intermediate | X | R¹ | R² | R⁴ | m.p. [° C.] |
|---|---|---|---|---|---|
| A1 | —Cl | —C₃H₇ | —CH₃ | —OCH₃ | 121-123 |
| A2 | —Cl | —C₂H₅ | —CH₃ | —OCH₃ | 148-150 |
| A3 | —Cl | —CH₃ | —CH₃ | —OCH₃ | 176-178 |
| A4 | —Cl | —C₆H₁₁ | —CH₃ | —OCH₃ | 211-213 |
| A5 | —Cl | —C₆H₁₃ | —CH₃ | —OCH₃ | 115-117 |
| A6 | —Cl | —C₅H₁₁ | —CH₃ | —OCH₃ | 110.5-113 |
| A7 | —Cl | —CH₂CH₂CF₃ | —CH₃ | —OCH₃ | 149-153 |
| A8 | —Cl | —(CH₂)₂C₆H₅ | —CH₃ | —OCH₃ | 130 |
| A9 | —Cl | —C₆H₅ | —CH₃ | —OCH₃ | 240-242 |
| A10 | —Cl | —C₆H₄(4-F) | —CH₃ | —OCH₃ | 256-258 |
| A11 | —Cl | —C₂H₅ | —CH₃ | —H | 117-120 |
| A12 | —Cl | —C₃H₇ | —CH₃ | —H | 138-140 |
| A13 | —Cl | —C₃H₇ | —H | —OCH₃ | 153-155 |
| A14 | —Cl | —CH(CH₃)₂ | —H | —OCH₃ | 162-164 |
| A15 | —Cl | —CH₃ | —H | —OCH₃ | 225-228 |
| A16 | —Cl | —H | —H | —H | 222-225 |
| A17 | —Cl | —H | —C₆H₅ | —OCH₃ | 168-171 |
| A18 | —Cl | —H | —CH₃ | —OCH₃ | 185-187 |
| A19 | —Cl | —C₃H₇ | —CH₃ | —CH₃ | 99-101 |
| A20 | —Cl | —C₂H₅ | —CH₃ | —N(C₂H₅)₂ | 145-150 |
| A21 | —Cl | —C₃H₇ | —CH₃ | 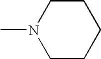 | |
| A22 | —Cl | —C₂H₅ | —CH₃ | 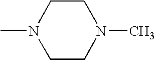 | 283-285 |
| A23 | —Cl | —C₂H₅ | —CH₃ | 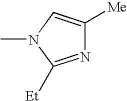 | 138-141 |
| A24 | —Cl | —C₃H₇ | —CH₃ | 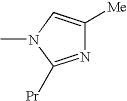 | 134-136 |

### Intermediate A 25

### 4-chloro-3-methyl-1-propyl-imidazo[1,5-a]pyrido[3,2-e]pyrazin-8-ol

2 g 4-chloro-8-methoxy-3-methyl-1-propyl-imidazo[1,5-a]pyrido[3,2-e]pyrazine was suspended in 50 ml dichloromethane. At 0-5° C. 3 ml bortribromide was added dropwise, followed by 1 h stirring at 0-5° C., 4 h stirring at room temperature, and standing over night. The reaction mixture was added slowly to a solution of 10 g potassium carbonate in 100 ml water. After stirring and constant pH>7 (adding 10% potassium carbonate solution) the precipitate was filtered off, and washed with water.

yield: 1.87 g

m.p.: 227-234° C. (EtOH)

Other intermediates A of formula (IV) can be prepared according to this procedure. Examples with X═Br were obtained with a period of 6 h heating to reflux. Some examples are the following:

| Intermediate | X | R¹ | R² | R⁴ | m.p. [° C.] |
|---|---|---|---|---|---|
| A25 | —Cl | —C₃H₇ | —CH₃ | —OH | 227-234 |
| A26 | —Br | —C₂H₅ | —CH₃ | —OH | >360° C. |
| | | | | | (x HBr) |
| A27 | —Br | —C₆H₁₁ | —CH₃ | —OH | 212-216 |

### Intermediate A28

### 4-chloro-8-difluoromethoxy-3-methyl-1-propyl-imidazo[1,5-a]pyrido[3,2-e]pyrazine

5.51 g (0.02 mol) 4-chloro-3-methyl-1-propyl-9H-imidazo[1,5-a]pyrido[3,2-e]pyrazin-8-ol and 2 g (0.05 mol) sodium hydroxide were dissolved in 20 ml dimethylformamide. After 10 min stirring 2.53 ml (0.03 mol) chlorodifluoroacetic acid was added dropwise. The mixture was heated 5 h at 150° C. bath temperature with stirring. After cooling the product was extracted with ethyl acetate (200 ml, 300 ml), the combined organic phases were washed with water (2×100 ml), the organic phase was dried over sodium sulfate, filtered off, and evaporated to dryness.

The obtained residue with 3 alkylated products was separated by preparative chromatography (silica gel, dichloromethane/methanol=9/1, v/v).

yield: 1.21 g

m.p.: 95-98° C.

### Example 1

### 4-amino-8-methoxy-3-methyl-1-propyl-imidazo[1,5-a]pyrido[3,2-e]pyrazine

10 g of intermediate A1 and 200 ml of an aqueous solution of NH₃ (32%) are mixed in an autoclave and heated up to 130° C. for 8 hours. The reaction mixture is diluted with 200 ml water. The precipitated reaction product is separated washed with water and dichloro methane and dried at reduced pressure.

Yield: 8.5 g

m.p.: 219-221° C.

The following examples are prepared using the same route of synthesis and reaction conditions like described above for example 1:

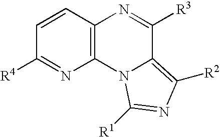

| Example | R¹ | R² | R³ | R⁴ | Fp [° C.] |
|---|---|---|---|---|---|
| 1 | —C₃H₇ | —CH₃ | —NH₂ | —OCH₃ | 219-221 |
| 2 | —C₂H₅ | —CH₃ | —NH₂ | —OCH₃ | 215-217 |
| 3 | —C₂H₅ | —CH₃ | —NH₂ | —H | 190-191 |
| 4 | —C₃H₇ | —CH₃ | —NH₂ | —H | 163-165 |
| 5 | —C₂H₅ | —CH₃ | —NH₂ | 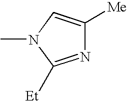 | 277-281 |
| 6 | —C₃H₇ | —CH₃ | —NH₂ | 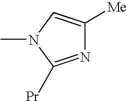 | 215-221 |
| 7 | —C₆H₁₃ | —CH₃ | —NH₂ | —OCH₃ | 167-169 |
| 8 | —CH₂CH₂CF₃ | —CH₃ | —NH₂ | —OCH₃ | 273-276 |
| 9 | —(CH₂)₂C₆H₅ | —CH₃ | —NH₂ | —OCH₃ | 198-200 |
| 10 | —C₆H₅ | —CH₃ | —NH₂ | —OCH₃ | 248-250 |
| 11 | —C₆H₄(2-Cl) | —CH₃ | —NH₂ | —OCH₃ | 248-250 |
| 12 | —C₆H₄(4-F) | —CH₃ | —NH₂ | —OCH₃ | 245-251 |
| 13 | —CH(CH₃)₂ | —H | —NH₂ | —OCH₃ | 277-279 |
| 14 | —H | —H | —NH₂ | —OCH₃ | 239-241 |
| 15 | —H | —C₆H₅ | —NH₂ | —OCH₃ | 252-253 |
| 16 | —C₃H₇ | —CH₃ | —NHCH₃ | —OCH₃ | 111-113 |
| 17 | —C₃H₇ | —CH₃ | —NHC₂H₅ | —OCH₃ | 140-142 |
| 18 | —C₂H₅ | —CH₃ | —NHCH₃ | —OCH₃ | 172-174 |
| 19 | —C₃H₇ | —CH₃ | —N(CH₃)₂ | —OCH₃ | 93-95 |
| 20 | —C₂H₅ | —CH₃ | —NHC₄H₉ | —OCH₃ | 62-65 |
| 21 | —C₂H₅ | —CH₃ | —NHCH₂C₆H₅ | —OCH₃ | 127-128 |
| 22 | —C₂H₅ | —CH₃ | 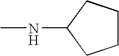 | —OCH₃ | 93-95 |
| 23 | —C₃H₇ | —CH₃ | 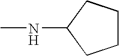 | —OCH₃ | 77-80 |
| 24 | —C₂H₅ | —CH₃ | 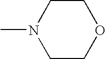 | —OCH₃ | 132-133 |
| 25 | —CH₂CH₂CF₃ | —CH₃ | 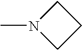 | —OCH₃ | 150-152 |
| 26 | —C₃H₇ | —CH₃ | 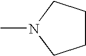 | —OCH₃ | 71-74 |
| 27 | —C₃H₇ | —CH₃ | 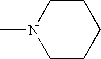 | —OCH₃ | 127-129 |
| 28 | —C₂H₅ | —CH₃ | 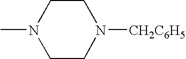 | —OCH₃ | 224-227 |
| 29 | —C₃H₇ | —CH₃ | 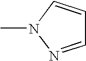 | —OCH₃ | 125-126 |
| 30 | —C₃H₇ | —CH₃ | 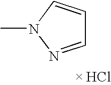 | —OCH₃ | 185-188 |
| 31 | —C₃H₇ | —CH₃ | 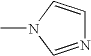 | —OCH₃ | 137-139 |
| 32 | —C₃H₇ | —CH₃ | 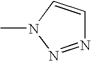 | —OCH₃ | 218-220 |
| 33 | —C₃H₇ | —CH₃ | 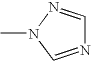 | —OCH₃ | 167-169 |
| 34 | —C₃H₇ | —CH₃ | 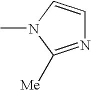 | —OCH₃ | 163-164 |
| 35 | —C₃H₇ | —CH₃ | 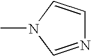 | —OH | 313-320 |

### Example 36

### 1-ethyl-4-(N-formyl-amino)-8-methoxy-3-methyl-imidazo[1,5-a]pyrido[3,2-e]pyrazine

A mixture of 2.1 ml of methane carboxylic acid and 5 ml of acetic acid anhydride is stirred at 60-70° C. for 1 hour. At room temperature 1 g of 4-amino-1-ethyl-8-methoxy-3-methyl-imidazo[1,5-a]pyrido[3,2-e]pyrazine (example 2) is added. After stirring for 5 hours at 30° C. the mixture is neutralized by addition of NaHCO₃ solution. The crude product is collected washed with water and dried at 40° C. For the final purification column chromatography is used (dichloro methane/methanol 3:1).

Yield: 0.6 g

m.p.: 206-208° C.

The following examples are prepared using the same route of synthesis and reaction conditions as described above for Example 26:

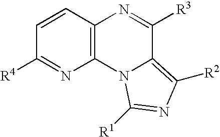

| Ex- | | | | | |
|---|---|---|---|---|---|
| am- | | | | | |
| ple | R¹ | R² | R³ | R⁴ | Fp [° C.] |
| 36 | —C₂H₅ | —CH₃ | —NHCH═O | —OCH₃ | 206-208 |
| 37 | —C₃H₇ | —CH₃ | —NHCH═O | —OCH₃ | 205-207 |
| 38 | —C₃H₇ | —CH₃ | —NH(C═O)CH₃ | —OCH₃ | 210-213 |
| 39 | —C₃H₇ | —CH₃ | —N[(C═O)CH₃]₂ | —OCH₃ | 135-138 |
| 40 | —C₂H₅ | —CH₃ | —NH(C═O)CH₃ | —OCH₃ | 199-202 |
| 41 | —C₂H₅ | —CH₃ | —N[(C═O)CH₃]₂ | —OCH₃ | 135-136 |
| 42 | —C₆H₅ | —CH₃ | —NH(C═O)CH₃ | —OCH₃ | 190-192 |
| 43 | —C₃H₇ | —CH₃ | —NH(C═O)C₂H₅ | —OCH₃ | 193-195 |
| 44 | —C₃H₇ | —CH₃ | 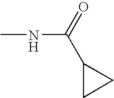 | —OCH₃ | 200-201 |

Surprisingly, the compounds of formula (II) are potent inhibitors of the enzyme PDE10. A substance is considered to effectively inhibit PDE10 if it has an IC₅₀ of less than 10 μM, preferably less than 1 μM.

Preparation and Characterization of PDE10

Phosphodiesterase isoenzyme 10 (PDE10) activity was determined in preparations of rat, pig and guinea pig striatum respectively. Striatum from male Wistar rats (180-200 g), male hybrid pigs (150 kg) and male guinea pigs (CRL (HA), 500 g) respectively were collected and frozen at −70° C.

In the prepared brain areas gene segments containing the catalytic domain of the PDE10 were amplified and sequenced. Therefore RNA from the frozen striatum of the different animals was isolated according to the instructions of the RNeasy kit (Qiagen; Hilden; Germany) and transcribed into cDNA using Oligo-Primer provided with the 1^{st} strand cDNA synthese kit for RT-PCR (Roche; Mannheim; Germany). These cDNA was used as template for the PCR-reaction to amplify the catalytic domain of the PDE10. For the PCR reaction Taq-Polymerase (Promega; Mannheim; Germany) was used. Therefore it was possible to clone the amplificates directly by TA-cloning in the pCR2.1 vector (Invitrogen; Karlsruhe; Germany). The cloning vector was transformed into E. colis (XL-2), replicated within the cells, prepared and the included gene sequence determined for the pig and the guinea pig.

The following primers were used for the PCR-reaction:

| | | |
|---|---|---|
| P1: | tgcatctacagggttaccatggagaa | (SEQ ID NO:1) |
| P2: | tatccctgcaggccttcagcagaggctct | (SEQ ID NO:2) |
| P3: | ttcacatggatatgcgacggtaccttct | (SEQ ID NO:3) |
| P4: | ctgtgaagaagaactatcggcgggttcctta | (SEQ ID NO:4) |

For the pig the priming was successful with P1 and P2. The following sequence (SEQ ID NO:5) was identified:

tgcatctacagggttaccatggagaagctgtcctaccacagcatttgtac
cgcggaagagtggcaaggcctcatgcgcttcaaccttcccgtccgtcttt
gcaaggagattgaattgttccacttcgacattggtccttttgaaaacatg
tggcctggaatctttgtctatatggttcatcgcttctgtgggacggcctg
ctttgagcttgaaaagctgtgtcgttttatcatgtctgtgaagaagaact
atcgtcgggttccttaccacaactggaagcacgcggtcacggtggcacac
tgcatgtacgccatcctccagaacagccacgggctcttcaccgacctcga
gcgcaaaggactgctaatcgcgtgtctgtgccacgacctggaccacaggg
gcttcagcaacagctacctgcagaaattcgaccaccccctggccgctctc
tactccacgcccaccatggagcagcaccacttctcccagaccgtgtccat
cctccagttggaagggcacaacatcttctccaccctgagctccagtgagt
acgagcaggtgcttgagatcatccgcaaagccatcattgccacagacctc
gctttgtactttggaaacaggaaacagttggaggagatgtaccagaccgg
atcgctaaaccttaataaccagtcacatagagaccgcgtcattggtttga
tgatgactgcctgtgatctctgttccgtgacaaaactgtggccagtaaca
aaactgacggcaaatgatatatatgcggaattctgggccgagggcgatga
ggtgaagaagctgggaatacagcctattcccatgatggacagagacaaga
aggacgaagtcccacaaggccagctcggattctacaacgcggtagctatc
ccctgctacaccaccctcacccagatcttcccgcccacagagcctcttct
gaaggcctgcagggata

For the guinea pig the priming was successful with P4 and P2 as well as for P2 and P3.

The following sequence (SEQ ID NO:6) was identified with P4 and P2:

ctgtgaagaagaactatcggcgggttccttaccacaactggaagcatgca
gtcacggtggcgcactgcatgtacgccatacttcaaaacaacaatggcct
cttcacagaccttgagcgcaaaggcctgctaattgcctgtctgtgccatg
acctggaccacaggggcttcagtaacagctacctgcagaaattcgaccac
cccctggctgcgttgtactccacctccaccatggagcaacaccacttctc
ccagacggtgttcatcctccagctggaaggacacaacatcttctccaccc
tgagctccagcgagtacgagcaggtgctggagatcatccgcaaagccatc
atcgccactgacctcgcactgtactttgggaacaggaagcagttggagga
gatgtaccagacagggtcgctgaacctcaataaccagtcccatcgagacc
gcgtcatcggcttgatgatgactgcctgcgatctttgctctgtgacgaaa
ctatggccagttacaaaattgacagcaaatgatatatatgcagagttctg
ggctgagggggatgagatgaagaagttggggatacagcccatccctatga
tggacagagacaagaaggatgaagtccctcaaggacagcttggattctac
aatgctgtggccatcccctgctataccaccctgacgcagatcctcccacc
cacagagcctctgctgaaggcctgcagggata

The following sequence (SEQ ID NO:7) was identified with P2 and P3:

tagagcctctgctgaaggcctgcagggataacctcaatcagtgggagaag
gtaattcgaggggaagagacagcaatgtggatttcaggcccagcaactag
caaaagcacatcagggaagccgaccaggaaggtcgatgactgatcctgag
gtgatgtctgcctagcaactgactcaacctgcttctgtgacttcgttctt
tttatttttatttttttaacggggtgaaaacctctctcagaaggtaccgt
cgcatatccatgtgaa

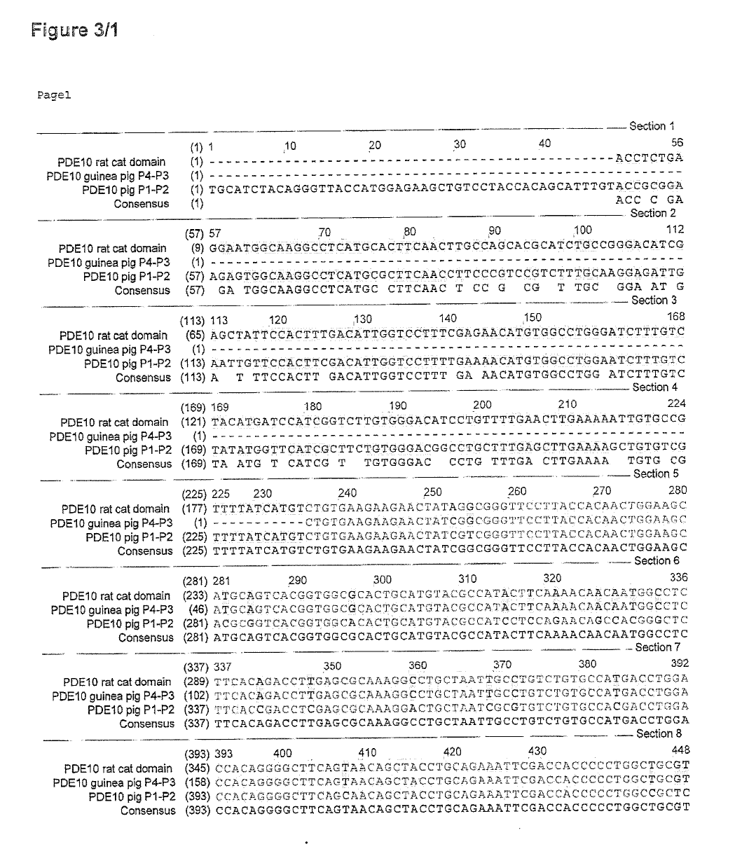

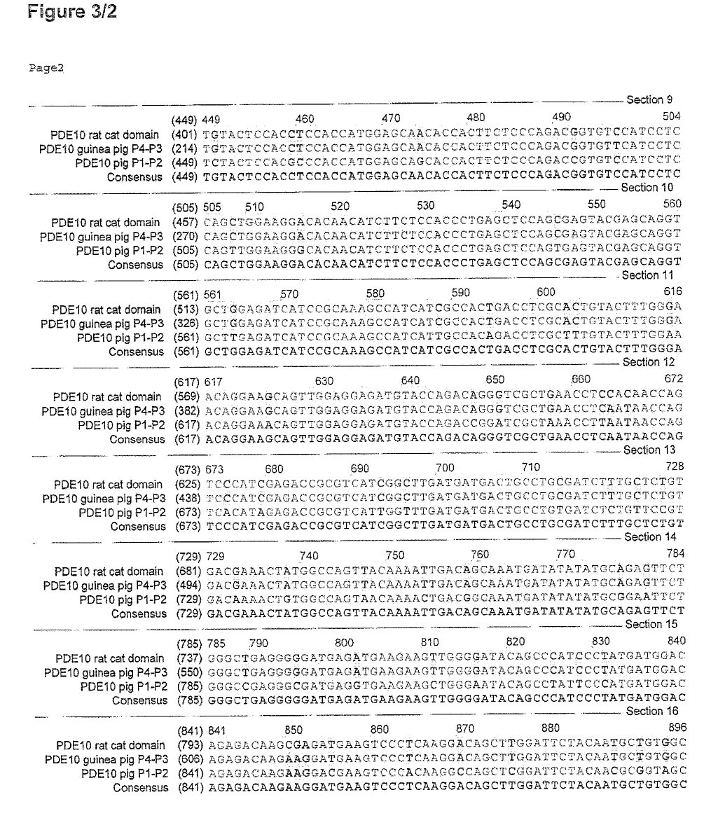

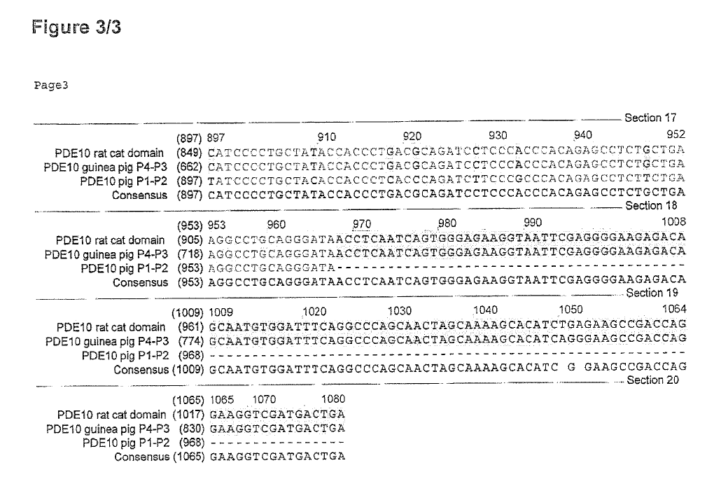

An alignment of the sequences showed a nearly complete accordance between the rat (published gene number NM_{—}022236 3437 bp; coding sequence: 281-2665; catalytic domain 1634-2665) and the guinea pig. More differences were detected between rat and pig. For the alignment only the coding areas were used. The gene alignment is shown in FIG. 3.

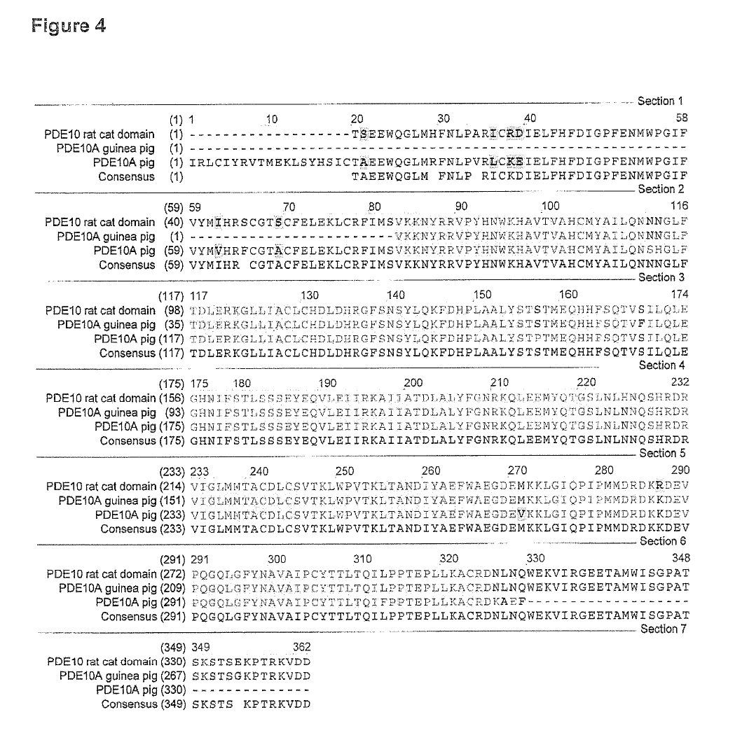

This results in the following differences in the protein sequences within the catalytic domain, as shown in a protein alignment (FIG. 4).

For the enzymatic testing of PDE10 activity 0.5 g of the isolated and frozen striatum was homogenised in 10 ml 50 mM Tris/Mg-buffer at 4° C. and centrifuged for one hour at 100000 g. The supernatant is called the cytosolic fraction and was removed and stored on ice. The pellet was resuspended in the same buffer, but containing 1% Triton and incubated for 45 min at 4° C. Both fractions were independently applied onto a 5 ml Hi Trap™ QHP column at the Äkta-FPLC. After washing the columns the bound PDE protein was eluted with an increasing sodium chloride gradient (0 mM-500 mM sodium chloride) in 50 mM Tris/Mg-buffer at 4° C. for the cytosolic fraction and in the presence of 1% Triton for the membrane fraction. The eluted and collected fractions were tested with 100 nM [³H]-cAMP for PDE10-activity in the presence and without a specific PDE-Inhibitor at a concentration, were a 100% inhibition is expected. The fractions with PDE10-activity were pooled and frozen in aliquots until use at −20° C.

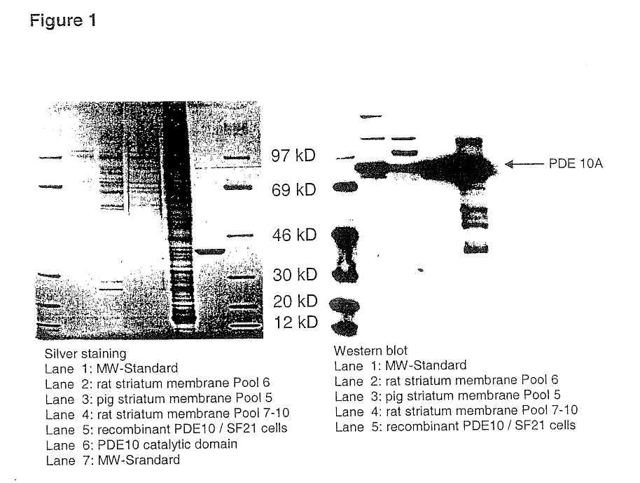

The pooled fractions from the FPLC were characterized by Western blot. It was shown that the PDE10A containing pooled fractions include a great number of other cellular proteins. Nevertheless PDE10 was detected with specific antibodies by Western blot clearly (FIG. 1).

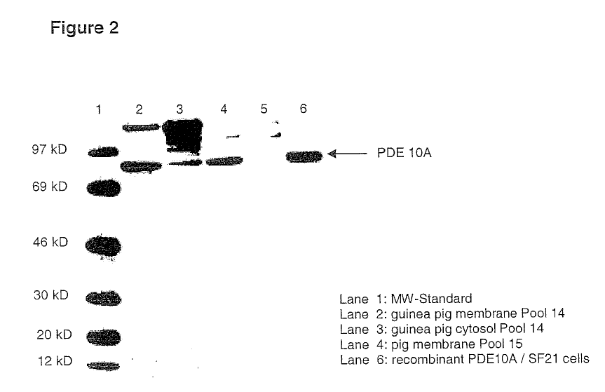

The protein was proven in the preparation of the striatum of the rat, the pig and the guinea pig. The main part of protein was found in the membrane fraction (FIG. 2).

Inhibition of PDE10

PDE10 activity was determined in a one step procedure in microtiterplates. The reaction mixture of 100 μl contained 50 mM Tris-HCl/5 mM MgCl₂ buffer (pH=7.4) (Sigma, Deisenhofen, Germany; Merck, Darmstadt, Germany) 0.1 μM [³H]-cAMP (Amersham, Buckinghamshire, UK) and the enzyme. Nonspecific activity was tested without the enzyme. The reaction was initiated by addition of the substrate solution and was carried out at 37° C. for 30 minutes. Enzymatic activity was stopped by addition of 25 μl YSi—SPA-beads (Amersham-Pharmacia). One hour later the mixture was measured in a liquid scintillation counter for microtiterplates (Microbeta Trilux). To pipette the incubation mixture a robot Biomek (Fa. Beckman) is used. The determined Km-values for the substrate cAMP is 78 nM for PDE10 from rat striatum, 88 nM for pig striatum and 66.7 nM for guinea pig striatum respectively. cGMP is the second substrate for PDE10, the Km values are 1800 nM, 2200 nM and 1700 nM for PDE10 from these species. For the test with cGMP 500 nM of this substrate was used. The optimal amount of enzyme in the assay has been determined and optimised for each enzyme preparation and substrate separately before using the enzyme in compound testing. For determination of IC₅₀ values the Hill-plot, 2-parameter-model, was used. Specific inhibitors of other PDE-Subtypes do not inhibit the PDE10 preparation significantly. Papaverine was used as the most common PDE10 inhibitor and inhibits the PDE10 with IC50 values of 142 nM, 110 nM and 77 nM for PDE10 from striatum of rat, pig and guinea pig respectively.

| | Inhibition of PDE10 from rat |
|---|---|
| Example | IC₅₀ [μM] |
| 1 | 0.006 |
| 3 | 0.043 |
| 4 | 0.057 |
| 10 | 0.005 |
| 36 | 0.241 |
| 37 | 0.050 |
| 40 | 0.220 |
| 42 | 0.095 |
| 43 | 2.410 |
| 44 | 2.180 |
| | Inhibition of PDE10 from pig |
| Example | IC₅₀ [μM] |
| 2 | 0.015 |
| 3 | 0.041 |
| 4 | 0.027 |
| 5 | 0.006 |
| 6 | 0.001 |
| 7 | 0.048 |
| 9 | 0.038 |
| 10 | 0.003 |
| 11 | 0.0005 |
| 12 | 0.006 |
| 26 | 0.137 |
| 27 | 0.302 |
| 29 | 0.199 |
| 30 | 0.155 |
| 31 | 0.009 |
| 33 | 0.025 |
| 34 | 0.395 |
| 35 | 0.086 |
| 36 | 0.080 |
| 37 | 0.029 |
| 42 | 0.041 |
| 43 | 0.896 |
| 44 | 0.671 |

| | Inhibition of PDE10 from guinea pig |
|---|---|
| Example | IC₅₀ [μM] |
| 3 | 0.037 |
| 11 | 0.001 |
| 31 | 0.011 |

Surprisingly, also the intermediates A of the synthesis of compounds of formula II are potent inhibitors of the enzyme PDE10.

| | Inhibition of PDE10 from rat |
|---|---|
| Intermediate | IC₅₀ [μM] |
| A1 | 0.008 |
| A2 | 0.023 |
| A11 | 0.171 |
| A14 | 0.237 |
| | Inhibition of PDE10 from pig |
| Intermediate | IC₅₀ [μM] |
| A1 | 0.004 |
| A2 | 0.017 |
| A4 | 0.002 |
| A5 | 0.071 |
| A6 | 0.056 |
| A7 | 0.034 |
| A9 | 0.004 |
| A11 | 0.097 |
| A12 | 0.038 |
| A13 | 0.053 |
| A14 | 0.128 |
| A19 | 0.009 |
| A20 | 0.011 |
| A21 | 0.005 |
| A22 | 0.052 |
| A23 | 0.003 |
| A24 | 0.002 |
| A25 | 0.063 |
| A26 | 0.046 |
| A28 | 0.008 |

The compounds of formula (II) show significant antipsychotic effects on the MK-801-induced hyperactivity and stereotyped sniffing, an animal model of psychosis.

Test Procedure:

Female Wistar rats (Crl: (WI) BR, Charles River, Sulzfeld, Germany) weighing 150 to 180 g were used for the MK-801-induced psychosis. Animals were housed under standard conditions in groups of five on a 12 h light/dark cycle (light on at 0600 h) with ad libitum access to food (Pellets, ssniff M/R 15, Spezialdiät GmbH, Soest/Westfalen) and water.

MK-801 (dizocilpine, MW 337.37) was obtained by Tocris, distributed by Biotrend Chemikalien GmbH, Köln, Germany.

Drug administration schedule/dosage:

| | | | number of | |
|---|---|---|---|---|
| | Dosage | pre-treatment | application | Route of |
| substance | [mg/kg] | [min] | [n] | administration |
| MK-801 | 0.1 | 10 | 1 | i.p. |
| Example 1 | 10, 30 | 30 | 1 | p.o. |
| Example 11 | 0.5, 1.0, | 30 | 1 | p.o. |
| | 2.5, 5.0, | | | |
| | 10 | | | |

Preparation of Compounds:

Compounds were freshly suspended in 0.5% hydroxyethylcellulose so that an administration volume of 0.5 ml/100 g was reached for each dose. Hydroxyethylcellulose was solved in distilled water.

MK-801 was dissolved in saline so that an administration volume of 0.5 ml/100 g was reached. The suspensions and solution were placed on a magnetic stirrer before and during dosing procedures.

The behaviour induced by the NMDA antagonist MK-801 is generally accepted as a rat model of psychosis. MK-801 induces stereotyped sniffing and hyperactivity in rats after intraperitoneal administration.

Locomotor activity of the rats was recorded by the MotiTest Apparatus (TSE, Bad Homburg, Germany). The test area consisted of a squared arena (45×45 cm) with protective plexiglass walls (20 cm of height) where rats could freely move. Horizontal movements were recorded by 32 infrared photocells arranged along the bottom of each wall of the arena. Activity [sec] was measured by the computer program “ActiMot” (TSE, Bad Homburg, Germany).

Stereotyped sniffing was scored by the experimenter every five minutes for one hour (12 intervals) according to the method described by Andiné et al. (1999). The scores of the 12 intervals were summed up at the end of the experiment.

| score | stereotyped sniffing |
|---|---|
| 0 | no stereotyped sniffing |
| 1 | discontinuous sniffing (free interval >5 s) |
| 2 | continuous sniffing |

The day of experiment the female rats were placed in the laboratory and received the test compound or vehicle at the appropriate time prior to test. MK-801 0.1 mg/kg was intraperitoneally administered 10 minutes prior to test.

At the beginning of the test the rats were placed in the centre of the squared arena of the MotiTest apparatus. Behaviour of the rats was recorded for one hour. After each run animals were removed and the boxes thoroughly cleaned and dried.

Statistics

Results were analysed by one way analysis of variance (ANOVA). Tukey test was used for individual comparison. P<0.05 was regarded as significant.

Results

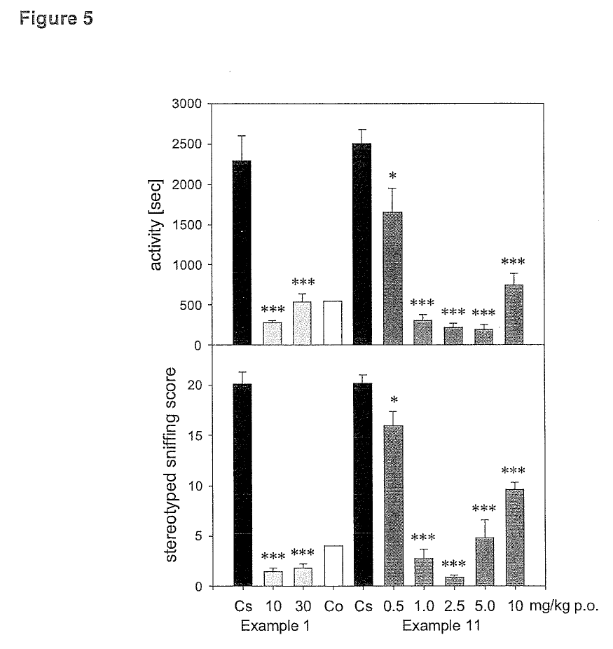

The results are shown in FIG. 5. MK-801 at 0.1 mg/kg i.p. was administered 10 min before testing. The compounds of Example 1 and 11 were administered 30 min prior to the test at the described doses. Activity and stereotyped sniffing was recorded for 1 h. Co=control without MK-801 stimulation, Cs=control with MK-801 stimulation. Significant to MK-801 stimulated control (=Cs): * p<0.05, *** p<0.001.

The compound of Example 1 significantly reversed MK-801-induced hyperactivity and stereotyped sniffing starting at 10 mg/kg p.o. The compound of Example 11 significantly reversed MK-801-induced hyperactivity and stereotyped sniffing starting at 0.5 mg/kg p.o. The results give evidence for the antipsychotic potential of the compounds.

1-28. (canceled)

29. A compound of formula (II) 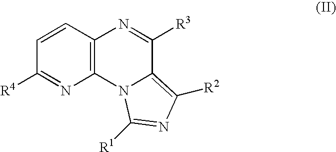
wherein R¹ and R² are independently selected from
H,
a cyclic radical,
C₁₋₈ alkyl or C₃₋₈ cycloalkyl, optionally mono- or polysubstituted with halo, OH, O—C₁₋₃ alkyl, or a cyclic radical,
C₂₋₈ alkenyl or C₃₋₈ cycloalkenyl, optionally mono- or polysubstituted with halo, OH, O—C₁₋₃ alkyl or a cyclic radical,
C₂-C₈ alkynyl, optionally mono- or polysubstituted with halo, OH, O—C₁-C₃-alkyl, or a cyclic radical,
a saturated, monounsaturated or polyunsaturated heterocycle with 5 to 15 ring atoms, optionally mono- or polysubstituted with halo, amino, C₁₋₃ alkylamino, di-C₁₋₃ alkylamino, nitro, C₁₋₃ alkyl, or O—C₁₋₃ alkyl, and
phenyl, optionally mono- or polysubstituted with halo, amino, C₁₋₃ alkylamino, di-C₁₋₃ alkylamino, nitro, C₁₋₃ alkyl, O—C₁₋₃ alkyl or a cyclic radical;
R³ is NH₂, NHR⁵ or NR⁵R⁶;
wherein R⁵ and R⁶ are independently selected from
a cyclic radical,
C₁₋₅ alkyl, optionally mono- or polysubstituted with halo, OH, O—C₁₋₃ alkyl or a cyclic radical,
aryl-C₁₋₅-alkyl wherein aryl is phenyl, optionally mono- or polysubstituted with halo, nitro, C₁₋₃ alkyl, or O—C₁₋₃ alkyl,
C═O)—C₁₋₅ alkyl optionally mono- or polysubstituted with halo, OH, O—C₁₋₃ alkyl or a cyclic radical, or
R⁵R⁶ together form a saturated or unsaturated five- or six-membered ring which can contain up to 3 heteroatoms, preferably N,N-oxide, S and O, optionally mono- or polysubstituted with halo, C₁₋₃ alkyl, O—C₁₋₃ alkyl or aryl-C₁₋₅-alkyl, wherein aryl is phenyl, optionally mono- or polysubstituted with halo, amino, C₁₋₃ alkylamino, di-C₁₋₃ alkylamino, nitro, C₁₋₃ alkyl, O—C₁₋₃ alkyl or a cyclic radical,
R⁴ is selected from
H,
halo,
a cyclic radical,
R⁷,
OH or OR⁷,
NH(C═O)—C₁₋₃ alkyl, optionally mono- or polysubstituted with halo, OH, O—C₁₋₃ alkyl or a cyclic radical or
NH₂, NHR⁷ or NR⁷R⁸,
wherein R⁷ and R⁸ are independently selected from
a cyclic radical,
C₁₋₆ alkyl or C₃₋₆ cycloalkyl, optionally mono- or polysubstituted with halo, OH, O—C₁₋₃ alkyl or a cyclic radical,
aryl-C₁₋₅-alkyl wherein aryl is phenyl, optionally mono- or polysubstituted with halo, amino, C₁₋₃ alkylamino, di-C₁₋₃ alkylamino, nitro, C₁₋₃ alkyl, OC₁₋₃ alkyl or a cyclic radical,
NR⁷R⁸ together form a saturated or unsaturated five-, six- or seven-membered ring which can contain up to 3 heteroatoms, preferably N including N-oxide, S and O, optionally mono- or polysubstituted with halo, C₁₋₃ alkyl, C₃₋₆ cycloalkyl, O—C₁₋₃ alkyl or aryl-C₁₋₅-alkyl, wherein aryl is phenyl, optionally mono- or polysubstituted with halo, amino, C₁₋₃ alkylamino, di-C₁₋₃ alkylamino, nitro, C₁₋₃ alkyl, O—C₁₋₃ alkyl or a cyclic radical,
or a pharmaceutically acceptable salt or derivative thereof.

30. The compounds of claim 29, wherein R¹ is selected from H, C₁₋₄ alkyl optionally mono- or polysubstituted with halo, OH, O—C₁₋₃ alkyl or a cyclic radical, phenyl, optionally mono- or polysubstituted with halo, amino, C₁₋₃ alkylamino, di-C₁₋₃ alkylamino, nitro, C₁₋₃ alkyl, O—C₁₋₃ alkyl or a cyclic radical.

31. The compound of claim 29, wherein R² is selected from H or C₁₋₄ alkyl optionally halogenated, particularly methyl or trifluoromethyl.

32. The compound of claim 29, wherein R³ is selected from NH₂, NHC₁₋₃ alkyl, optionally mono- or polysubstituted with halo, OH or O—C₁₋₃ alkyl, or
NH(C═O)—C₁₋₃ alkyl, optionally mono- or polysubstituted with halo, OH or O—C₁₋₃ alkyl.

33. The compound of claim 29, wherein R³ is selected from cyclopropyl, cyclobutyl, tetrahydropyrrolyl, pyrrolyl, pyrazolyl, imidazolyl, 1,2,3-triazolyl, 1,2,4-triazolyl, piperidinyl, morpholinyl, piperazinyl, optionally substituted with C₁₋₃ alkyl, optionally mono- or polysubstituted with halo, OH or O—C₁₋₃ alkyl, or arylalkyl, wherein aryl is phenyl, optionally mono- or polysubstituted with halo, amino, C₁₋₃ alkylamino, di-C₁₋₃ alkylamino, nitro, C₁₋₃ alkyl, O—C₁₋₃ alkyl or a cyclic radical.

34. The compound of claim 29, wherein R⁴ is selected from OH or O—C₁₋₃ alkyl, optionally mono- or polysubstituted with halo, OH or O—C₁₋₃ alkyl, NHC₁₋₃ alkyl, optionally mono- or polysubstituted with halo, OH or O—C₁₋₃ alkyl, or NH benzyl, wherein the phenyl group is phenyl, optionally mono- or polysubstituted with halo, amino, C₁₋₃ alkylamino, di-C₁₋₃ alkylamino, nitro, C₁₋₃ alkyl, OC₁₋₃ alkyl or a cyclic radical.

35. The compound of claim 29, wherein R⁴ is selected from cyclopropyl, cyclobutyl, tetrahydropyrrolyl, pyrrolyl, pyrazolyl, imidazolyl, 1,2,3-triazolyl, 1,2,4-triazolyl, piperidinyl, morpholinyl, piperazinyl, optionally substituted with C₁₋₃ alkyl, optionally mono- or polysubstituted with halo, OH or O—C₁₋₃ alkyl, or arylalkyl, wherein aryl is phenyl, optionally mono- or polysubstituted with halo, amino, C₁₋₃ alkylamino, di-C₁₋₃ alkylamino, nitro, C₁₋₃ alkyl, O—C₁₋₃ alkyl or a cyclic radical.

36. The compound of claim 29 selected from the group consisting of
4-amino-8-methoxy-3-methyl-1-propyl-imidazo[1,5-a]pyrido[3,2-e]pyrazine;
4-amino-1-ethyl-8-methoxy-3-methyl-imidazo[1,5-a]pyrido[3,2-e]pyrazine;
4-amino-1-ethyl-3-methyl-imidazo[1,5-a]pyrido[3,2-e]pyrazine;
4-amino-3-methyl-1-propyl-imidazo[1,5-a]pyrido[3,2-e]pyrazine;
4-amino-1-ethyl-8-(2-ethyl-4-methyl-imidazol-1-yl)-3-methyl-imidazo[1,5-a]pyrido[3,2-e]pyrazine;
4-amino-3-methyl-1-propyll-8-(2-propyl-4-methyl-imidazol-1-yl)-imidazo[1,5-a]pyrido[3,2-e]pyrazine;
4-amino-1-hexyl-8-methoxy-3-methyl-imidazo[1,5-a]pyrido[3,2-e]pyrazine;
4-amino-8-methoxy-3-methyl-1-(3,3,3-trifluoropropyl)-imidazo[1,5-a]pyrido[3,2-e]pyrazine;
4-amino-8-methoxy-3-methyl-1-phenethyl-imidazo[1,5-a]pyrido[3,2-e]pyrazine;
4-amino-8-methoxy-3-methyl-1-phenyl-imidazo[1,5-a]pyrido[3,2-e]pyrazine;
4-amino-1-(2-chloro-phenyl)-8-methoxy-3-methyl-imidazo[1,5-a]pyrido[3,2-e]pyrazine;
4-amino-1-(4-fluoro-phenyl)-8-methoxy-3-methyl-imidazo[1,5-a]pyrido[3,2-e]pyrazine;
4-amino-1-isopropyl-8-methoxy-imidazo[1,5-a]pyrido[3,2-e]pyrazine;
4-amino-8-methoxy-imidazo[1,5-a]pyrido[3,2-e]pyrazine;
4-amino-8-methoxy-3-phenyl-imidazo[1,5-a]pyrido[3,2-e]pyrazine;
4-(N-methyl-amino)-8-methoxy-3-methyl-1-propyl-imidazo[1,5-a]pyrido[3,2-e]pyrazine;
4-(N-ethyl-amino)-8-methoxy-3-methyl-1-propyl-imidazo[1,5-a]pyrido[3,2-e]pyrazine;
4-(N-methyl-amino)-1-ethyl-8-methoxy-3-methyl-imidazo[1,5-a]pyrido[3,2-e]pyrazine;
4-(N,N-dimethyl-amino)-8-methoxy-3-methyl-1-propyl-imidazo[1,5-a]pyrido[3,2-e]pyrazine;
4-(N-butyl-amino)-1-ethyl-8-methoxy-3-methyl-imidazo[1,5-a]pyrido[3,2-e]pyrazine;
4-(N-benzyl-amino)-1-ethyl-8-methoxy-3-methyl-imidazo[1,5-a]pyrido[3,2-e]pyrazine;
4-(N-cyclopentyl-amino)-1-ethyl-8-methoxy-3-methyl-imidazo[1,5-a]pyrido[3,2-e]pyrazine;
4-(N-cyclopentyl-amino)-8-methoxy-3-methyl-1-propyl-imidazo[1,5-a]pyrido[3,2-e]pyrazine;
1-ethyl-8-methoxy-3-methyl-4-morpholino-imidazo[1,5-a]pyrido[3,2-e]pyrazine;
4-azetidine-8-methoxy-3-methyl-1-(3,3,3-trifluoropropyl)-imidazo[1,5-a]pyrido[3,2-e]pyrazine;
8-methoxy-3-methyl-1-propyl-4-pyrrolidino-imidazo[1,5-a]pyrido[3,2-e]pyrazine;
8-methoxy-3-methyl-4-piperidino-1-propyl-imidazo[1,5-a]pyrido[3,2-e]pyrazine;
1-ethyl-8-methoxy-3-methyl-4-(4-phenylpiperazino)-imidazo[1,5-a]pyrido[3,2-e]pyrazine;
8-methoxy-3-methyl-1-propyl-4-(pyrazol-1-yl)-imidazo[1,5-a]pyrido[3,2-e]pyrazine;
8-methoxy-3-methyl-1-propyl-4-(pyrazol-1-yl)-imidazo[1,5-a]pyrido[3,2-e]pyrazine hydro chloride;
4-(imidazol-1-yl)-8-methoxy-3-methyl-1-propyl-imidazo[1,5-a]pyrido[3,2-e]pyrazine;
8-methoxy-3-methyl-1-propyl-4-(1,2,3-triazol-1-yl)-imidazo[1,5-a]pyrido[3,2-e]pyrazine;
8-methoxy-3-methyl-1-propyl-4-(1,2,4-triazol-1-yl)-imidazo[1,5-a]pyrido[3,2-e]pyrazine;
8-methoxy-3-methyl-4-(2-methyl-imidazol-1-yl)-1-propyl-imidazo[1,5-a]pyrido[3,2-e]pyrazine;
4-(imidazol-1-yl)-3-methyl-1-propyl-imidazo[1,5-a]pyrido[3,2-e]pyrazine-8-ol;
1-ethyl-4-(N-formyl-amino)-8-methoxy-3-methyl-imidazo[1,5-a]pyrido[3,2-e]pyrazine;
4-(N-formyl-amino)-8-methoxy-3-methyl-1-propyl-imidazo[1,5-a]pyrido[3,2-e]pyrazine;
4-(N-acetyl-amino)-8-methoxy-3-methyl-1-propyl-imidazo[1,5-a]pyrido[3,2-e]pyrazine;
4-(N,N-diacetyl-amino)-8-methoxy-3-methyl-1-propyl-imidazo[1,5-a]pyrido[3,2-e]pyrazine;
4-(N-acetyl-amino)-1-ethyl-8-methoxy-3-methyl-imidazo[1,5-a]pyrido[3,2-e]pyrazine;
4-(N,N-diacetyl-amino)-1-ethyl-8-methoxy-3-methyl-imidazo[1,5-a]pyrido[3,2-e]pyrazine;
4-(N-acetyl-amino)-8-methoxy-3-methyl-1-phenyl-imidazo[1,5-a]pyrido[3,2-e]pyrazine;
8-methoxy-3-methyl-4-(N-propionyl-amino)-1-propyl-imidazo[1,5-a]pyrido[3,2-e]pyrazine;
4-(N-cyclopropylcarboxy-amino)-8-methoxy-3-methyl-1-propyl-imidazo[1,5-a]pyrido[3,2-e]pyrazine;
or a pharmaceutically acceptable salt or derivative thereof.

37. A method for preparing a compound of claim 29 comprising
i. reacting a compound of formula (III): 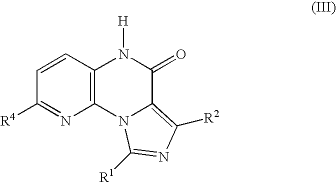
with a halogenating agent, to obtain a compound of formula (IV): 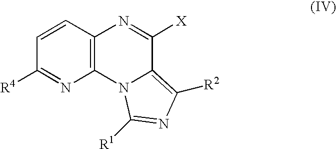
wherein X is Cl or Br;
ii. reacting the compound of formula (IV) with an amine HR³ to obtain the compound of formula (II) and
iii. optionally reacting s compound of formula (II), wherein R⁵ and R⁶ are H with an acylating agent.

38. The method of claim 37, wherein the halogenating agent is a chlorinating or brominating agent or the acylating agent is a carboxylic acid chloride or a carboxylic acid anhydride.

39. A pharmaceutical composition comprising a compound of claim 29, or a compound of formula (IV) 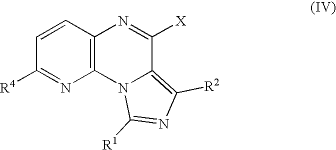
wherein X is Cl or Br;
optionally together with pharmaceutically acceptable carrier, diluent or adjuvant.

40. A method for treating or preventing a disorder caused by, associated with or accompanied by phosphodiesterase 10 hyperactivity or a disorder in which inhibiting phosphodiesterase 10 is of value by administering a therapeutically effective amount of the compound of claim 29 or a compound of formula (IV): 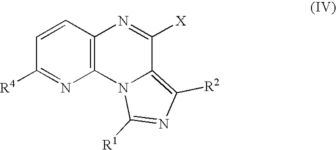
wherein X is Cl or Br; to a subject in need thereof.

41. A method for treating or preventing a central nervous system disorder comprising administering a therapeutically effective amount of the compound of claim 29 or a compound of formula (IV): 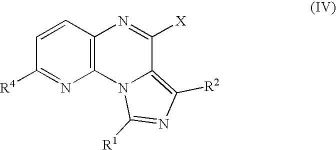
wherein X is Cl or Br; to a subject in need thereof.

42. A method of treating a neurological and psychiatric disorder including schizophrenia and other psychotic disorders; mood disorders; neurotic, stress-related and somatoform disorders including anxiety disorders; eating disorders; sexual dysfunction comprising excessive sexual drive; disorders of adult personality and behaviour; disorders usually first diagnosed in infancy, childhood and adolescence; mental retardation; disorders of psychological development; and a disorder having a symptom of cognitive deficiency in a mammal, including a human; factitious disorder by administering a therapeutically effective amount of the compound of claim 29 or a compound of formula (IV): 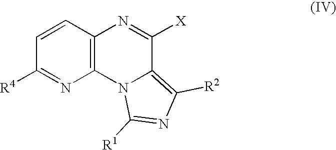
wherein X is Cl or Br; to a subject in need thereof.

43. The method of claim 42, wherein the schizophrenia and other psychotic disorder are continuous or episodic schizophrenia of different type selected from the group consisting of paranoid, hebephrenic, catatonic, undifferentiated, residual, and a schizophreniform disorder; a schizotypal disorder selected from the group consisting of borderline, latent, prepsychotic, prodromal, pseudoneurotic pseudopsychopathic schizophrenia and schizotypal personality disorder; a persistent delusional disorder; an acute, transient or persistent psychotic disorder; induced delusional disorder; a schizoaffective disorder of different type selected from a manic, depressive or mixed type; a puerperal psychosis and unspecified nonorganic psychosis.

44. A method comprising treating or preventing an affective disorder selected from the group consisting of a manic episode associated to bipolar disorder and a single manic episode, hypomania, mania with psychotic symptoms; a bipolar affective disorder, a bipolar affective disorder with a current hypomanic or manic episode with or without a psychotic symptom; a depressive disorder, a single episode or recurrent major depressive disorder, a depressive disorder with postpartum onset, a depressive disorder with a psychotic symptom; a persistent affective disorder cyclothymia, dysthymia; or premenstrual dysphoric disorder by administering a therapeutically effective amount of the compound of claim 29 or a compound of formula (IV): 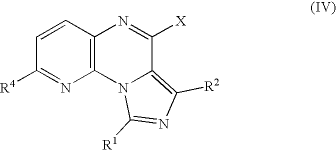
wherein X is Cl or Br; to a subject in need thereof.

45. A method of treating or preventing a neurotic, stress-related or somatoform disorder that is a phobic anxiety disorder, for instance agoraphobia and social phobia primarily but not exclusively related to psychosis; another anxiety disorder such as a panic disorder and a general anxiety disorder; obsessive compulsive disorder; a reaction to sever stress and adjustment disorder; a dissociative disorder or depersonalization-derealization syndrome by administering a therapeutically effective amount of the compound of claim 29 or a compound of formula (IV): 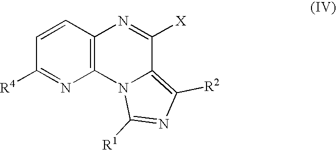
wherein X is Cl or Br; to a subject in need thereof.

46. A method comprising treating or preventing a disorder of adult personality and behavior which are specific personality disorder of the paranoid, schizoid, schizotypal, antisocial, borderline, histrionic, narcissistic, avoidant, dissocial, emotionally unstable, anankastic, anxious and dependent type; a mixed personality disorder; a habit and impulse disorder; or a disorder of sexual preference by administering a therapeutically effective amount of the compound of claim 29 or a compound of formula (IV): 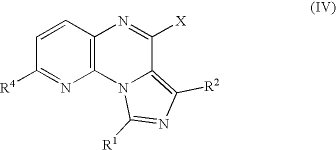
wherein X is Cl or Br; to a subject in need thereof.

47. A method comprising treating or preventing a hyperkinetic disorder, attentional deficit/hyperactivity disorder (AD/HD), a conduct disorder; a mixed disorder of conduct and emotional disorder; a nonorganic enuresis, a nonorganic encopresis; a stereotyped movement disorder, attention deficit disorder without hyperactivity, excessive masturbation, nail-biting, nose-picking and thumb-sucking; a disorder of psychological development particularly schizoid disorder of childhood and a pervasive development disorder by administering a therapeutically effective amount of the compound of claim 29 or a compound of formula (IV): 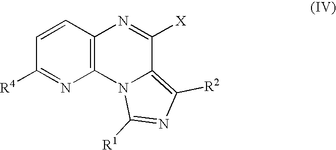
wherein X is Cl or Br; to a subject in need thereof.

48. A method comprising treating or preventing a developmental disorder of speech and language, a developmental disorder of scholastic skills, which disorders are predominantly diagnosed in infancy, childhood and adolescence by administering a therapeutically effective amount of the compound of claim 29 or a compound of formula (IV): 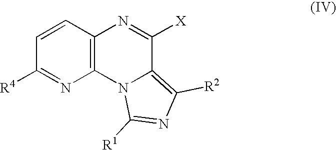
wherein X is Cl or Br; to a subject in need thereof.

49. A method comprising treating or preventing a symptom cognitive deficiency that is a cognitive deficit primarily but not exclusively related to psychosis; an age-associated memory impairment, Parkinson's disease, Alzheimer's disease, multi infarct dementia, Lewis body dementia, stroke, frontotemporal dementia, progressive supranuclear palsy Huntington's disease and in HIV disease, cerebral trauma, drug abuse and a mild cognitive disorder by administering a therapeutically effective amount of the compound of claim 29 or a compound of formula (IV): 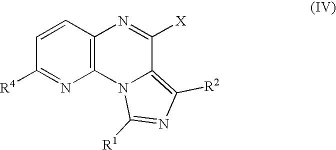
wherein X is Cl or Br; to a subject in need thereof.

50. A method comprising treating or preventing a movement disorder with a malfunction of basal ganglia which are a different subtype of dystonia selected from the group consisting of a focal dystonia, a multiple-focal dystonia or a segmental dystonia, a torsion dystonia, hemispheric, generalized and tardive dyskinesia, a drug induced dyskenesia, an akathisia, or a dyskinesia selected from Huntington's disease, Parkinson's disease, Lewis body disease, restless leg syndrome or PLMS by administering a therapeutically effective amount of the compound of claim 29 or a compound of formula (IV): 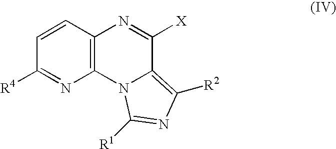
wherein X is Cl or Br; to a subject in need thereof.

51. A method comprising treating or preventing an organic disorder selected from the group consisting of a symptomatic mental disorder, an organic delusional disorder, presenil or senile psychosis associated to dementia, a psychosis in epilepsy, Parkinson's disease, an organic and symptomatic psychosis; delirium; infective psychosis; personality disorder or a behavioural disorders due to brain disease, damage and dysfunction by administering a therapeutically effective amount of the compound of claim 29 or a compound of formula (IV): 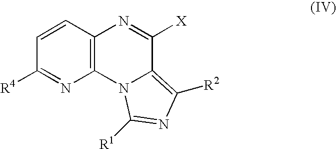
wherein X is Cl or Br; to a subject in need thereof.

52. A method comprising treating or preventing a psychotic disorder and residual and late-onset psychotic disorder induced by alcohol, opioids, cannabinoids, cocaine, hallucinogens, other stimulants, including caffeine, volatile solvent or a psychoactive compound by administering a therapeutically effective amount of the compound of claim 29 or a compound of formula (IV): 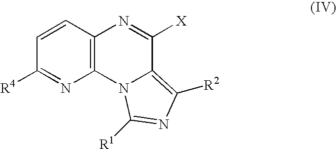
wherein X is Cl or Br; to a subject in need thereof.

55. A method for improving learning and memory capacities in a mammal by administering a therapeutically effective amount of the compound of claim 29 or a compound of formula (IV): 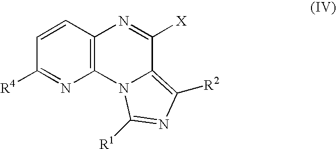
wherein X is Cl or Br; to a subject in need thereof.

56. The method of claim 55, wherein the subject is a mammal selected from a human or an animal.

57. A pharmaceutical composition comprising at least one compound of claim 29 or a compound of formula (IV) 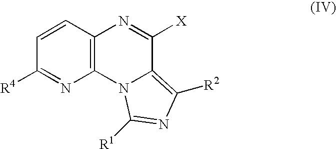
wherein X is Cl or Br and at least one further pharmaceutically active compound.

58. A kit comprising at least one compound of claim 29 or a compound of formula (IV) 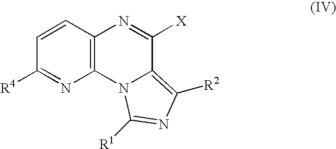
wherein X is Cl or Br and at least one further pharmaceutically active compound.

59. The composition of claim 57, wherein the further active compound is a therapeutically active compound useful in the treatment of central nervous system disorders which is not based on PDE 10 inhibition.

60. The kit of claim 58, wherein the further active compound is a therapeutically active compound useful in the treatment of central nervous system disorders which is not based on PDE 10 inhibition.

61. The method of claim 38, wherein the brominating agent is POCl₃, PCl₃, PCl₅, SOCl₂, POBr₃, PBr₃ or PBr₅.
